SEQUENCE LISTING (27 sequences)

SEQ ID NO: 1 - protein, 65 aa, Zymobacter palmae
MYTVGMYLAERLAQIGLKHHFAVAGDYNLVLLDQLLLNKDMEQVYCCNELNCGFSAEGYARARGA

SEQ ID NO: 2 - protein, 42 aa, Zymobacter palmae
HIMMVGDGSFQLTAQEVAQMIRYEIPVIIFLINNRGYVIEIA

SEQ ID NO: 3 - protein, 25 aa, Geobacillus kaustophilus
MSGSLMLIEALKEEKVEVIFGYPGG

SEQ ID NO: 4 - protein, 24 aa, Geobacillus kaustophilus
GFQMTFQELSVIQELRLPIKIVIV

SEQ ID NO: 5 - protein, 555 aa, Artificial Sequence
  Hybrid pyruvate decarboxylase N1
MSGSLMLIEALKEEKVEHHFAVAGDYNLVLLDQLLLNKDMEQVYCCNELNCGFSAEGYARARGAAAAIVTFSVGAISAMNAIGGAYAENLPVILISGSPNTNDYGTGHILHHTIGTTDYNYQLEMVKHVTCARESIVSAEEAPAKIDHVIRTALRERKPAYLEIACNVAGAECVRPGPINSLLRELEVDQTSVTAAVDAAVEWLQDRQNVVMLVGSKLRAAAAEKQAVALADRLGCAVTIMAAEKGFFPEDHPNFRGLYWGEVSSEGAQELVENADAILCLAPVFNDYATVGWNSWPKGDNVMVMDTDRVTFAGQSFEGLSLSTFAAALAEKAPSRPATTQGTQAPVLGIEAAEPNAPLTNDEMTRQIQSLITSDTTLTAETGDSWFNASRMPIPGGARVELEMQWGHIGWSVPSAFGNAVGSPERRHIMMVGDGSFQLTAQEVAQMIRYEIPVIIFLINNRGYVIEIAIHDGPYNYIKNWNYAGLIDVFNDEDGHGLGLKASTGAELEGAIKKALDNRRGPTLIECNIAQDDCTETLIAWGKRVAATNSRKPQA

SEQ ID NO: 6 - protein, 555 aa, Artificial Sequence
  Hybrid pyruvate decarboxylase N2
MSGSLMLIEALKEEKVEVIFGYPGGYNLVLLDQLLLNKDMEQVYCCNELNCGFSAEGYARARGAAAAIVTFSVGAISAMNAIGGAYAENLPVILISGSPNTNDYGTGHILHHTIGTTDYNYQLEMVKHVTCARESIVSAEEAPAKIDHVIRTALRERKPAYLEIACNVAGAECVRPGPINSLLRELEVDQTSVTAAVDAAVEWLQDRQNVVMLVGSKLRAAAAEKQAVALADRLGCAVTIMAAEKGFFPEDHPNFRGLYWGEVSSEGAQELVENADAILCLAPVFNDYATVGWNSWPKGDNVMVMDTDRVTFAGQSFEGLSLSTFAAALAEKAPSRPATTQGTQAPVLGIEAAEPNAPLTNDEMTRQIQSLITSDTTLTAETGDSWFNASRMPIPGGARVELEMQWGHIGWSVPSAFGNAVGSPERRHIMMVGDGSFQLTAQEVAQMIRYEIPVIIFLINNRGYVIEIAIHDGPYNYIKNWNYAGLIDVFNDEDGHGLGLKASTGAELEGAIKKALDNRRGPTLIECNIAQDDCTETLIAWGKRVAATNSRKPQA

SEQ ID NO: 7 - protein, 555 aa, Artificial Sequence
  Hybrid pyruvate decarboxylase N1C
MSGSLMLIEALKEEKVEHHFAVAGDYNLVLLDQLLLNKDMEQVYCCNELNCGFSAEGYARARGAAAAIVTFSVGAISAMNAIGGAYAENLPVILISGSPNTNDYGTGHILHHTIGTTDYNYQLEMVKHVTCARESIVSAEEAPAKIDHVIRTALRERKPAYLEIACNVAGAECVRPGPINSLLRELEVDQTSVTAAVDAAVEWLQDRQNVVMLVGSKLRAAAAEKQAVALADRLGCAVTIMAAEKGFFPEDHPNFRGLYWGEVSSEGAQELVENADAILCLAPVFNDYATVGWNSWPKGDNVMVMDTDRVTFAGQSFEGLSLSTFAAALAEKAPSRPATTQGTQAPVLGIEAAEPNAPLTNDEMTRQIQSLITSDTTLTAETGDSWFNASRMPIPGGARVELEMQWGHIGWSVPSAFGNAVGSPERRHIMMVGDGGFQMTFQELSVIQELRLPIKIVIVNNRGYVIEIAIHDGPYNYIKNWNYAGLIDVFNDEDGHGLGLKASTGAELEGAIKKALDNRRGPTLIECNIAQDDCTETLIAWGKRVAATNSRKPQA

SEQ ID NO: 8 - protein, 555 aa, Artificial Sequence
  Hybrid pyruvate decarboxylase N2C
MSGSLMLIEALKEEKVEVIFGYPGGYNLVLLDQLLLNKDMEQVYCCNELNCGFSAEGYARARGAAAAIVTFSVGAISAMNAIGGAYAENLPVILISGSPNTNDYGTGHILHHTIGTTDYNYQLEMVKHVTCARESIVSAEEAPAKIDHVIRTALRERKPAYLEIACNVAGAECVRPGPINSLLRELEVDQTSVTAAVDAAVEWLQDRQNVVMLVGSKLRAAAAEKQAVALADRLGCAVTIMAAEKGFFPEDHPNFRGLYWGEVSSEGAQELVENADAILCLAPVFNDYATVGWNSWPKGDNVMVMDTDRVTFAGQSFEGLSLSTFAAALAEKAPSRPATTQGTQAPVLGIEAAEPNAPLTNDEMTRQIQSLITSDTTLTAETGDSWFNASRMPIPGGARVELEMQWGHIGWSVPSAFGNAVGSPERRHIMMVGDGGFQMTFQELSVIQELRLPIKIVIVNNRGYVIEIAIHDGPYNYIKNWNYAGLIDVFNDEDGHGLGLKASTGAELEGAIKKALDNRRGPTLIECNIAQDDCTETLIAWGKRVAATNSRKPQA

SEQ ID NO: 9 - protein, 556 aa, Artificial Sequence
  Hybrid pyruvate decarboxylase C
MYTVGMYLAERLAQIGLKHHFAVAGDYNLVLLDQLLLNKDMEQVYCCNELNCGFSAEGYARARGAAAAIVTFSVGAISAMNAIGGAYAENLPVILISGSPNTNDYGTGHILHHTIGTTDYNYQLEMVKHVTCARESIVSAEEAPAKIDHVIRTALRERKPAYLEIACNVAGAECVRPGPINSLLRELEVDQTSVTAAVDAAVEWLQDRQNVVMLVGSKLRAAAAEKQAVALADRLGCAVTIMAAEKGFFPEDHPNFRGLYWGEVSSEGAQELVENADAILCLAPVFNDYATVGWNSWPKGDNVMVMDTDRVTFAGQSFEGLSLSTFAAALAEKAPSRPATTQGTQAPVLGIEAAEPNAPLTNDEMTRQIQSLITSDTTLTAETGDSWFNASRMPIPGGARVELEMQWGHIGWSVPSAFGNAVGSPERRHIMMVGDGGFQMTFQELSVIQELRLPIKIVIVNNRGYVIEIAIHDGPYNYIKNWNYAGLIDVFNDEDGHGLGLKASTGAELEGAIKKALDNRRGPTLIECNIAQDDCTETLIAWGKRVAATNSRKPQA

SEQ ID NO: 10 - DNA, 1668 bp, Artificial Sequence
  Hybrid pyruvate decarboxylase N1
atgagcggttcgctgatgctcattgaagcgctcaaggaagaaaaagtcgaacaccactttgccgtggccggtgactacaacctggtgttgcttgatcagctcctgctgaacaaagacatggagcaggtctactgctgtaacgaacttaactgcggctttagcgccgaaggttacgctcgtgcacgtggtgccgccgctgccatcgtcacgttcagcgtaggtgctatctctgcaatgaacgccatcggtggcgcctatgcagaaaacctgccggtcatcctgatctctggctcaccgaacaccaatgactacggcacaggccacatcctgcaccacaccattggtactactgactataactatcagctggaaatggtaaaacacgttacctgcgcacgtgaaagcatcgtttctgccgaagaagcaccggcaaaaatcgaccacgtcatccgtacggctctacgtgaacgcaaaccggcttatctggaaatcgcatgcaacgtcgctggcgctgaatgtgttcgtccgggcccgatcaatagcctgctgcgtgaactcgaagttgaccagaccagtgtcactgccgctgtagatgccgccgtagaatggctgcaggaccgccagaacgtcgtcatgctggtcggtagcaaactgcgtgccgctgccgctgaaaaacaggctgttgccctagcggaccgcctgggctgcgctgtcacgatcatggctgccgaaaaaggcttcttcccggaagatcatccgaacttccgcggcctgtactggggtgaagtcagctccgaaggtgcacaggaactggttgaaaacgccgatgccatcctgtgtctggcaccggtattcaacgactatgctaccgttggctggaactcctggccgaaaggcgacaatgtcatggtcatggacaccgaccgcgtcactttcgcaggacagtccttcgaaggtctgtcattgagcaccttcgccgcagcactggctgagaaagcaccttctcgcccggcaacgactcaaggcactcaagcaccggtactgggtattgaggccgcagagcccaatgcaccgctgaccaatgacgaaatgacgcgtcagatccagtcgctgatcacttccgacactactctgacagcagaaacaggtgactcttggttcaacgcttctcgcatgccgattcctggcggtgctcgtgtcgaactggaaatgcaatggggtcatatcggttggtccgtaccttctgcattcggtaacgccgttggttctccggagcgtcgccacatcatgatggtcggtgatggctctttccagctgactgctcaagaagttgctcagatgatccgctatgaaatcccggtcatcatcttcctgatcaacaaccgcggttacgtcatcgaaatcgctatccatgacggcccttacaactacatcaaaaactggaactacgctggcctgatcgacgtcttcaatgacgaagatggtcatggcctgggtctgaaagcttctactggtgcagaactagaaggcgctatcaagaaagcactcgacaatcgtcgcggtccgacgctgatcgaatgtaacatcgctcaggacgactgcactgaaaccctgattgcttggggtaaacgtgtagcagctaccaactctcgcaaaccacaagcgtaa

SEQ ID NO: 11 - DNA, 1668 bp, Artificial Sequence
  Hybrid pyruvate decarboxylase N2
atgagcggttcgctgatgctcattgaagcgctcaaggaagaaaaagtcgaagtcatcttcggctatccgggtggttacaacctggtgttgcttgatcagctcctgctgaacaaagacatggagcaggtctactgctgtaacgaacttaactgcggctttagcgccgaaggttacgctcgtgcacgtggtgccgccgctgccatcgtcacgttcagcgtaggtgctatctctgcaatgaacgccatcggtggcgcctatgcagaaaacctgccggtcatcctgatctctggctcaccgaacaccaatgactacggcacaggccacatcctgcaccacaccattggtactactgactataactatcagctggaaatggtaaaacacgttacctgcgcacgtgaaagcatcgtttctgccgaagaagcaccggcaaaaatcgaccacgtcatccgtacggctctacgtgaacgcaaaccggcttatctggaaatcgcatgcaacgtcgctggcgctgaatgtgttcgtccgggcccgatcaatagcctgctgcgtgaactcgaagttgaccagaccagtgtcactgccgctgtagatgccgccgtagaatggctgcaggaccgccagaacgtcgtcatgctggtcggtagcaaactgcgtgccgctgccgctgaaaaacaggctgttgccctagcggaccgcctgggctgcgctgtcacgatcatggctgccgaaaaaggcttcttcccggaagatcatccgaacttccgcggcctgtactggggtgaagtcagctccgaaggtgcacaggaactggttgaaaacgccgatgccatcctgtgtctggcaccggtattcaacgactatgctaccgttggctggaactcctggccgaaaggcgacaatgtcatggtcatggacaccgaccgcgtcactttcgcaggacagtccttcgaaggtctgtcattgagcaccttcgccgcagcactggctgagaaagcaccttctcgcccggcaacgactcaaggcactcaagcaccggtactgggtattgaggccgcagagcccaatgcaccgctgaccaatgacgaaatgacgcgtcagatccagtcgctgatcacttccgacactactctgacagcagaaacaggtgactcttggttcaacgcttctcgcatgccgattcctggcggtgctcgtgtcgaactggaaatgcaatggggtcatatcggttggtccgtaccttctgcattcggtaacgccgttggttctccggagcgtcgccacatcatgatggtcggtgatggctctttccagctgactgctcaagaagttgctcagatgatccgctatgaaatcccggtcatcatcttcctgatcaacaaccgcggttacgtcatcgaaatcgctatccatgacggcccttacaactacatcaaaaactggaactacgctggcctgatcgacgtcttcaatgacgaagatggtcatggcctgggtctgaaagcttctactggtgcagaactagaaggcgctatcaagaaagcactcgacaatcgtcgcggtccgacgctgatcgaatgtaacatcgctcaggacgactgcactgaaaccctgattgcttggggtaaacgtgtagcagctaccaactctcgcaaaccacaagcgtaa

SEQ ID NO: 12 - DNA, 1668 bp, Artificial Sequence
  Hybrid pyruvate decarboxylase N1C
atgagcggttcgctgatgctcattgaagcgctcaaggaagaaaaagtcgaacaccactttgccgtggccggtgactacaacctggtgttgcttgatcagctcctgctgaacaaagacatggagcaggtctactgctgtaacgaacttaactgcggctttagcgccgaaggttacgctcgtgcacgtggtgccgccgctgccatcgtcacgttcagcgtaggtgctatctctgcaatgaacgccatcggtggcgcctatgcagaaaacctgccggtcatcctgatctctggctcaccgaacaccaatgactacggcacaggccacatcctgcaccacaccattggtactactgactataactatcagctggaaatggtaaaacacgttacctgcgcacgtgaaagcatcgtttctgccgaagaagcaccggcaaaaatcgaccacgtcatccgtacggctctacgtgaacgcaaaccggcttatctggaaatcgcatgcaacgtcgctggcgctgaatgtgttcgtccgggcccgatcaatagcctgctgcgtgaactcgaagttgaccagaccagtgtcactgccgctgtagatgccgccgtagaatggctgcaggaccgccagaacgtcgtcatgctggtcggtagcaaactgcgtgccgctgccgctgaaaaacaggctgttgccctagcggaccgcctgggctgcgctgtcacgatcatggctgccgaaaaaggcttcttcccggaagatcatccgaacttccgcggcctgtactggggtgaagtcagctccgaaggtgcacaggaactggttgaaaacgccgatgccatcctgtgtctggcaccggtattcaacgactatgctaccgttggctggaactcctggccgaaaggcgacaatgtcatggtcatggacaccgaccgcgtcactttcgcaggacagtccttcgaaggtctgtcattgagcaccttcgccgcagcactggctgagaaagcaccttctcgcccggcaacgactcaaggcactcaagcaccggtactgggtattgaggccgcagagcccaatgcaccgctgaccaatgacgaaatgacgcgtcagatccagtcgctgatcacttccgacactactctgacagcagaaacaggtgactcttggttcaacgcttctcgcatgccgattcctggcggtgctcgtgtcgaactggaaatgcaatggggtcatatcggttggtccgtaccttctgcattcggtaacgccgttggttctccggagcgtcgccacatcatgatggtcggcgacggcggcttccaaatgacgttccaagaactgtcggtcatccaggagctacggctgccgatcaaaatcgtcatcgtcaacaaccgcggttacgtcatcgaaatcgctatccatgacggcccttacaactacatcaaaaactggaactacgctggcctgatcgacgtcttcaatgacgaagatggtcatggcctgggtctgaaagcttctactggtgcagaactagaaggcgctatcaagaaagcactcgacaatcgtcgcggtccgacgctgatcgaatgtaacatcgctcaggacgactgcactgaaaccctgattgcttggggtaaacgtgtagcagctaccaactctcgcaaaccacaagcgtaa

SEQ ID NO: 13 - DNA, 1668 bp, Artificial Sequence
  Hybrid pyruvate decarboxylase N2C
atgagcggttcgctgatgctcattgaagcgctcaaggaagaaaaagtcgaagtcatcttcggctatccgggtggttacaacctggtgttgcttgatcagctcctgctgaacaaagacatggagcaggtctactgctgtaacgaacttaactgcggctttagcgccgaaggttacgctcgtgcacgtggtgccgccgctgccatcgtcacgttcagcgtaggtgctatctctgcaatgaacgccatcggtggcgcctatgcagaaaacctgccggtcatcctgatctctggctcaccgaacaccaatgactacggcacaggccacatcctgcaccacaccattggtactactgactataactatcagctggaaatggtaaaacacgttacctgcgcacgtgaaagcatcgtttctgccgaagaagcaccggcaaaaatcgaccacgtcatccgtacggctctacgtgaacgcaaaccggcttatctggaaatcgcatgcaacgtcgctggcgctgaatgtgttcgtccgggcccgatcaatagcctgctgcgtgaactcgaagttgaccagaccagtgtcactgccgctgtagatgccgccgtagaatggctgcaggaccgccagaacgtcgtcatgctggtcggtagcaaactgcgtgccgctgccgctgaaaaacaggctgttgccctagcggaccgcctgggctgcgctgtcacgatcatggctgccgaaaaaggcttcttcccggaagatcatccgaacttccgcggcctgtactggggtgaagtcagctccgaaggtgcacaggaactggttgaaaacgccgatgccatcctgtgtctggcaccggtattcaacgactatgctaccgttggctggaactcctggccgaaaggcgacaatgtcatggtcatggacaccgaccgcgtcactttcgcaggacagtccttcgaaggtctgtcattgagcaccttcgccgcagcactggctgagaaagcaccttctcgcccggcaacgactcaaggcactcaagcaccggtactgggtattgaggccgcagagcccaatgcaccgctgaccaatgacgaaatgacgcgtcagatccagtcgctgatcacttccgacactactctgacagcagaaacaggtgactcttggttcaacgcttctcgcatgccgattcctggcggtgctcgtgtcgaactggaaatgcaatggggtcatatcggttggtccgtaccttctgcattcggtaacgccgttggttctccggagcgtcgccacatcatgatggtcggcgacggcggcttccaaatgacgttccaagaactgtcggtcatccaggagctacggctgccgatcaaaatcgtcatcgtcaacaaccgcggttacgtcatcgaaatcgctatccatgacggcccttacaactacatcaaaaactggaactacgctggcctgatcgacgtcttcaatgacgaagatggtcatggcctgggtctgaaagcttctactggtgcagaactagaaggcgctatcaagaaagcactcgacaatcgtcgcggtccgacgctgatcgaatgtaacatcgctcaggacgactgcactgaaaccctgattgcttggggtaaacgtgtagcagctaccaactctcgcaaaccacaagcgtaa

SEQ ID NO: 14 - DNA, 1671 bp, Artificial Sequence
  Hybrid pyruvate decarboxylase C
atgtataccgttggtatgtacttggcagaacgcctagcccagatcggcctgaaacaccactttgccgtggccggtgactacaacctggtgttgcttgatcagctcctgctgaacaaagacatggagcaggtctactgctgtaacgaacttaactgcggctttagcgccgaaggttacgctcgtgcacgtggtgccgccgctgccatcgtcacgttcagcgtaggtgctatctctgcaatgaacgccatcggtggcgcctatgcagaaaacctgccggtcatcctgatctctggctcaccgaacaccaatgactacggcacaggccacatcctgcaccacaccattggtactactgactataactatcagctggaaatggtaaaacacgttacctgcgcacgtgaaagcatcgtttctgccgaagaagcaccggcaaaaatcgaccacgtcatccgtacggctctacgtgaacgcaaaccggcttatctggaaatcgcatgcaacgtcgctggcgctgaatgtgttcgtccgggcccgatcaatagcctgctgcgtgaactcgaagttgaccagaccagtgtcactgccgctgtagatgccgccgtagaatggctgcaggaccgccagaacgtcgtcatgctggtcggtagcaaactgcgtgccgctgccgctgaaaaacaggctgttgccctagcggaccgcctgggctgcgctgtcacgatcatggctgccgaaaaaggcttcttcccggaagatcatccgaacttccgcggcctgtactggggtgaagtcagctccgaaggtgcacaggaactggttgaaaacgccgatgccatcctgtgtctggcaccggtattcaacgactatgctaccgttggctggaactcctggccgaaaggcgacaatgtcatggtcatggacaccgaccgcgtcactttcgcaggacagtccttcgaaggtctgtcattgagcaccttcgccgcagcactggctgagaaagcaccttctcgcccggcaacgactcaaggcactcaagcaccggtactgggtattgaggccgcagagcccaatgcaccgctgaccaatgacgaaatgacgcgtcagatccagtcgctgatcacttccgacactactctgacagcagaaacaggtgactcttggttcaacgcttctcgcatgccgattcctggcggtgctcgtgtcgaactggaaatgcaatggggtcatatcggttggtccgtaccttctgcattcggtaacgccgttggttctccggagcgtcgccacatcatgatggtcggcgacggcggcttccaaatgacgttccaagaactgtcggtcatccaggagctacggctgccgatcaaaatcgtcatcgtcaacaaccgcggttacgtcatcgaaatcgctatccatgacggcccttacaactacatcaaaaactggaactacgctggcctgatcgacgtcttcaatgacgaagatggtcatggcctgggtctgaaagcttctactggtgcagaactagaaggcgctatcaagaaagcactcgacaatcgtcgcggtccgacgctgatcgaatgtaacatcgctcaggacgactgcactgaaaccctgattgcttggggtaaacgtgtagcagctaccaactctcgcaaaccacaagcgtaa

SEQ ID NO: 15 - protein, 63 aa, Geobacillus kaustophilus
MSGSLMLIEALKEEKVEVIFGYPGGAVLPLYDELYKAGVFHVLTRHEQGAIHAAEGYARISGK

SEQ ID NO: 16 - protein, 65 aa, Zymobacter palmae
MYTVGTYLAERLVQIGLKHHFAVAGDYNLVLLDNLLLNKNMEQVYCCNELNCGFSAEGYARAKGA

SEQ ID NO: 17 - protein, 50 aa, Zymobacter palmae
MYTVGMYLAERLAQIGLKHHFAVAGDYNLVLLDQLLLNKDMEQVYCCNEL

SEQ ID NO: 18 - protein, 49 aa, Artificial Sequence
  Pyruvate decarboxylase hybrid 1 and 3
MSGSLMLIEALKEEKVEHHFAVAGDYNLVLLDQLLLNKDMEQVYCCNEL

SEQ ID NO: 19 - protein, 49 aa, Artificial Sequence
  Pyruvate decarboxylase hybrid 2 and 4
MSGSLMLIEALKEEKVEVIFGYPGGYNLVLLDQLLLNKDMEQVYCCNEL

SEQ ID NO: 20 - protein, 42 aa, Zymobacter palmae; identical to SEQ ID NO: 2

SEQ ID NO: 21 - protein, 42 aa, Artificial Sequence
  Pyruvate decarboxylase hybrid 3 and 4
HIMMVGDGGFQMTFQELSVIQELRLPIKIVIVNNRGYVIEIA

SEQ ID NO: 22 - protein, 64 aa, Artificial Sequence
  Pyruvate decarboxylase hybrid 1
MSGSLMLIEALKEEKVEHHFAVAGDYNLVLLDQLLLNKDMEQVYCCNELNCGFSAEGYARARGA

SEQ ID NO: 23 - protein, 64 aa, Artificial Sequence
  Pyruvate decarboxylase hybrid 2
MSGSLMLIEALKEEKVEVIFGYPGGYNLVLLDQLLLNKDMEQVYCCNELNCGFSAEGYARARGA

SEQ ID NO: 24 - protein, 64 aa, Artificial Sequence; identical to SEQ ID NO: 22
  Pyruvate decarboxylase hybrid 3

SEQ ID NO: 25 - protein, 64 aa, Artificial Sequence; identical to SEQ ID NO: 23
  Pyruvate decarboxylase hybrid 4

SEQ ID NO: 26 - protein, 556 aa, Zymobacter palmae
MYTVGMYLAERLAQIGLKHHFAVAGDYNLVLLDQLLLNKDMEQVYCCNELNCGFSAEGYARARGAAAAIVTFSVGAISAMNAIGGAYAENLPVILISGSPNTNDYGTGHILHHTIGTTDYNYQLEMVKHVTCARESIVSAEEAPAKIDHVIRTALRERKPAYLEIACNVAGAECVRPGPINSLLRELEVDQTSVTAAVDAAVEWLQDRQNVVMLVGSKLRAAAAEKQAVALADRLGCAVTIMAAEKGFFPEDHPNFRGLYWGEVSSEGAQELVENADAILCLAPVFNDYATVGWNSWPKGDNVMVMDTDRVTFAGQSFEGLSLSTFAAALAEKAPSRPATTQGTQAPVLGIEAAEPNAPLTNDEMTRQIQSLITSDTTLTAETGDSWFNASRMPIPGGARVELEMQWGHIGWSVPSAFGNAVGSPERRHIMMVGDGSFQLTAQEVAQMIRYEIPVIIFLINNRGYVIEIAIHDGPYNYIKNWNYAGLIDVFNDEDGHGLGLKASTGAELEGAIKKALDNRRGPTLIECNIAQDDCTETLIAWGKRVAATNSRKPQA

SEQ ID NO: 27 - DNA, 1671 bp, Zymobacter palmae
atgtataccgttggtatgtacttggcagaacgcctagcccagatcggcctgaaacaccactttgccgtggccggtgactacaacctggtgttgcttgatcagctcctgctgaacaaagacatggagcaggtctactgctgtaacgaacttaactgcggctttagcgccgaaggttacgctcgtgcacgtggtgccgccgctgccatcgtcacgttcagcgtaggtgctatctctgcaatgaacgccatcggtggcgcctatgcagaaaacctgccggtcatcctgatctctggctcaccgaacaccaatgactacggcacaggccacatcctgcaccacaccattggtactactgactataactatcagctggaaatggtaaaacacgttacctgcgcacgtgaaagcatcgtttctgccgaagaagcaccggcaaaaatcgaccacgtcatccgtacggctctacgtgaacgcaaaccggcttatctggaaatcgcatgcaacgtcgctggcgctgaatgtgttcgtccgggcccgatcaatagcctgctgcgtgaactcgaagttgaccagaccagtgtcactgccgctgtagatgccgccgtagaatggctgcaggaccgccagaacgtcgtcatgctggtcggtagcaaactgcgtgccgctgccgctgaaaaacaggctgttgccctagcggaccgcctgggctgcgctgtcacgatcatggctgccgaaaaaggcttcttcccggaagatcatccgaacttccgcggcctgtactggggtgaagtcagctccgaaggtgcacaggaactggttgaaaacgccgatgccatcctgtgtctggcaccggtattcaacgactatgctaccgttggctggaactcctggccgaaaggcgacaatgtcatggtcatggacaccgaccgcgtcactttcgcaggacagtccttcgaaggtctgtcattgagcaccttcgccgcagcactggctgagaaagcaccttctcgcccggcaacgactcaaggcactcaagcaccggtactgggtattgaggccgcagagcccaatgcaccgctgaccaatgacgaaatgacgcgtcagatccagtcgctgatcacttccgacactactctgacagcagaaacaggtgactcttggttcaacgcttctcgcatgccgattcctggcggtgctcgtgtcgaactggaaatgcaatggggtcatatcggttggtccgtaccttctgcattcggtaacgccgttggttctccggagcgtcgccacatcatgatggtcggtgatggctctttccagctgactgctcaagaagttgctcagatgatccgctatgaaatcccggtcatcatcttcctgatcaacaaccgcggttacgtcatcgaaatcgctatccatgacggcccttacaactacatcaaaaactggaactacgctggcctgatcgacgtcttcaatgacgaagatggtcatggcctgggtctgaaagcttctactggtgcagaactagaaggcgctatcaagaaagcactcgacaatcgtcgcggtccgacgctgatcgaatgtaacatcgctcaggacgactgcactgaaaccctgattgcttggggtaaacgtgtagcagctaccaactctcgcaaaccacaagcgtaa

(19) United States
(12) Patent Application Publication
(10) Pub. No.: US 2015/0104850 A1
(43) Pub. Date: Apr. 16, 2015

(54) PROTEIN MANIPULATION

(71) Applicant: ReBio Technologies Limited, Cranleigh, Surrey (GB)

(72) Inventors: David Jonathan Leak, Bath (GB); Jeremy Bartosiak-Jentys, Bath (GB)

(73) Assignee: ReBioTechnologies Limted, Cranleigh, Surrey (GB)

(21) Appl. No.: 14/402,226

(22) PCT Filed: May 20, 2013
(86) PCT No.: PCT/GB2013/051303

§ 371 Date: Nov. 19, 2014

(30) Foreign Application Priority Data
May 22, 2012 (GB) 1209041.1

(51) Int. Cl. C12N 9/88

(52) U.S. Cl. CPC C12N 9/88; C12Y 401/01001
USPC 435/232

(57) ABSTRACT

A method of improving the folding of an enzyme comprising a thiamine pyrophosphate (TPP) binding domain, the method comprising: providing a nucleic acid encoding the enzyme comprising a TPP binding domain, in which one or more of the TPP binding domains in the enzyme monomer are replaced with a TPP binding domain from a thermostable TPP-binding protein, and expressing the nucleic acid under conditions that allow expression and folding of the enzyme. The enzyme may be pyruvate decarboxylase.

The invention relates to methods for improving the thermostability and folding properties of enzymes which contain thiamine pyrophosphate (TPP) binding domains, such as keto-acid decarboxylases, and uses thereof.

The listing or discussion of an apparently prior-published document in this specification should not necessarily be taken as an acknowledgement that the document is part of the state of the art or is common general knowledge.

The finite nature of fossil fuels dictates that we must look for sustainable renewable sources of liquid fuels and chemicals. Technologies based on starch derived from grain crops are inefficient in terms of their overall energy balance and greenhouse gas balance (Farrell et al, 2006). Consequently, there is a major thrust towards production from lignocellulosic (LC) feedstocks, either purpose-grown or as agricultural or municipal wastes (Ragauskas et al, 2006). This opens up the range of organisms that may usefully be considered for production purposes. Fermentation of glucose (from starch) or sucrose to ethanol has naturally focused on organisms such as Saccharomyces cerevisiae or Zymomonas mobilis, because of their high ethanol tolerance. However, they have a very restricted substrate range so where utilisation of LC-derived carbohydrate is considered, rather than express multiple different catabolic enzymes in these hosts, alternative more versatile organisms have been considered (Zhou et al, 2001; Shaw et al, 2008). Additionally, where products other than ethanol are the target, the advantages of S. cerevisiae and Z. mobilis diminish (van Haveren et al, 2008).

Thermophilic organisms are especially useful in fermenting LC-derived carbohydrates. Enzyme hydrolysis of LC-derived carbohydrates is typically done at around 55° C., so, for a thermophilic process running at 60-65° C., this mixture can be transferred directly into the reactor, retaining process heat and allowing many of the enzymes to continue working during the fermentation.

One such candidate thermophilic organism is the facultatively-anaerobic Gram-positive thermophile, Geobacillus thermoglucosidasius. Improved genetic tools have allowed precise gene deletions and promoter insertions to be made that redirect the natural mixed-acid fermentation to an ethanol pathway, involving anaerobic flux through pyruvate dehydrogenase (PDH) in this organism (Cripps et al, 2009). The fermentation pathway in G. thermoglucosidasius is shown in FIG. 21, and ethanologenic engineering strategies are shown in FIG. 22.

Although G. thermoglucosidasius is not as ethanol tolerant as S. cerevisiae and Z. mobilis, it can naturally transport and metabolise the major pentose monomers found in hemicelluloses. Furthermore, it can use cellobiose and short-chain xylans, as well as hexoses and pentoses, as substrates, which generates major process economies for the utilisation of LC feedstocks. Typically, these are subjected to physical and/or chemical pre-treatment (steam explosion, weak acid, alkali etc) to provide access to the cellulose and residual hemicelluloses, followed by an enzymatic treatment with a commercial “cellulase” mixture (Yang & Wyman, 2008). For strains that can only metabolise monomers, this clearly needs to achieve complete hydrolysis, whereas with G. thermoglucosidasius a cheaper partial hydrolysis is sufficient. This significantly reduces the cost compared to complete hydrolysis.

Geobacillus spp. can be particularly useful organisms for industrial processes as they grow rapidly at 40-70° C., which is compatible with typical process operations. Moreover, growth at ˜70° C. allows ethanol removal by gas stripping.

Pyruvate decarboxylase is an enzyme which converts pyruvate to acetaldehyde and carbon dioxide. Together with an alcohol dehydrogenase which reduces the acetaldehyde to ethanol it is part of the fermentation pathway in yeast that produces ethanol. Although it is common in eukaryotes, it is not commonly found in prokaryotes.

WO 03/025117 describes the cloning and sequencing of PDC genes from various bacteria, and their use for fermenting ethanol from sugars.

Both S. cerevisiae and bacteria of the genus Zymomonas (e.g., Z. mobilis, Z. palmae) can produce ethanol from pyruvate via pyruvate decarboxylase (PDC) and alcohol dehydrogenase. This combination of enzymes has been transferred as an operon into a number of other mesophiles to make them ethanologenic (US 2005/0158836). However, this strategy could not be used with G. thermoglucosidasius because no thermophilic PDC is available. While use of the PDH pathway is an alternative option, it may cause unexpected physiological consequences, so a dedicated PDC pathway might be preferable. Hence there is a need for thermophilic keto-acid decarboxylase enzymes, such as PDC, that are active in thermophiles and can be used in a thermophilic reactor process.

The inventors have now developed a method to achieve this aim based on alterations in the thiamine pyrophosphate (TPP) domains. Specifically, the inventors have shown that it is possible to make a more thermostable PDC enzyme by replacing one or more TPP binding domains in PDC with TPP binding domains from a thermophilic protein.

Thus, a first aspect of the invention provides a method of improving the folding of an enzyme comprising a thiamine pyrophosphate (TPP) binding domain, the method comprising:
providing a nucleic acid encoding the enzyme comprising a TPP binding domain, in which one or more of the TPP binding domains in the enzyme monomer are replaced with a TPP binding domain from a thermostable TPP-binding protein, and
expressing the nucleic acid under conditions that allow expression and folding of the enzyme.

By an “enzyme comprising a TPP binding domain” we include any protein that contains a TPP binding domain, and which possess at least detectable levels of enzymic activity. Since TPP binding domains are highly conserved between proteins, proteins containing them are well known in the art and it is possible to define a TPP binding enzyme family (see FIG. 18). Typically, such enzymes are ones that can catalyse the decarboxylation of keto-acids. The enzyme may be one that catalyses a reaction where the final product is the decarboxylated acid, or the enzyme may be one that decarboxylates a keto-acid to form an intermediate which is the substrate for a further reaction that is catalysed by the enzyme.

It may be preferred if the enzyme is any of a keto-acid decarboxylase enzyme, a pyruvate decarboxylase, a keto-isovalerate decarboxylase, an alpha-ketoacid dehydrogenase, a branched chain amino acid dehydrogenase, a transketolase, a 2-hydroxyphytanoyl-CoA lyase, an alpha-ketoacid ferredoxin oxidoreductase, a glyoxylate carboligase, an oxalyl-CoA decarboxylase, an acetolactate synthase, an alpha-ketoacid oxidase, a sulfoacetaldehyde acetyltransferase, a 2-succinyl-5-enolpyruvyl-6-hydroxy-3-cyclohexene-1-carboxylate synthase, a pyruvate synthase, an epi-inositol hydrolase, a malonic semialdehyde oxidative decarboxylase, a pyruvate:flavodoxin oxidoreductase, a 3D-(3,5/4)-trihydroxycyclohexane-1,2-dione hydrolase, a 2-oxoglutarate synthase, a 2-succinyl-6-hydroxy-2,4-cyclohexadiene-1-carboxylic acid synthase, a phosphonopyruvate decarboxylase, a sulfopyruvate decarboxylase, a phenylglyoxylate:acceptor oxidoreductase, and a myo-inositol catabolism protein lolD.

In an embodiment, the enzyme is a keto-acid decarboxylase (e.g. an alpha keto-acid decarboxylase) such as any of oxalyl-CoA decarboxylase, indolepyruvate decarboxylase, malonic semialdehyde oxidative decarboxylase, phosphonopyruvate decarboxylase, sulfopyruvate decarboxylase, benzoylformate decarboxylase, 2-oxoglutarate decarboxylase, or phenylpyruvate decarboxylase.

In a particular embodiment, the enzyme is a pyruvate decarboxylase (PDC) (see e.g. Conway et al (1987); gi|48660|emb|CAA42157.1|; and http://www.ncbi.nlm.nih.gov/protein/AAA27696.2).

In another embodiment, the enzyme that comprises a TPP binding domain is a keto-isovalerate decarboxylase (see e.g. de la Plaza et al (2004); gi|51870502|emb|CAG342261|).

In an embodiment of the invention, it may be preferred that the enzyme comprising a TPP binding domain (e.g. a keto-acid decarboxylase, such as a PDC) is not one that is derived from a thermophile. Thus, typically, the enzyme is derived from a mesophile. By mesophile we include organisms that grow optimally at temperatures below 50° C., and so include bacteria as well as eukaryotes such as yeast, fungi, animals and plants. The mesophile may be a bacteria of the genus Zymomonas, such as Z. mobilis or Z. palmae, or a bacteria of the genus Acetobacter, Plantomyces, Prevotella, Acinetobacter, Gluconacetobacter or Sarcina.

Alternatively, the enzyme comprising a TPP binding domain may be one derived from a thermophile. For example, it may be desirable to modify such an enzyme so that it can be expressed and folded at even higher temperatures than those at which it is natively expressed and folded.

It may be preferred if the enzyme comprising a TPP binding domain (e.g., a keto-acid decarboxylase, such as a PDC) is one that is known to be thermostable after it has been folded (e.g. it may have a Tₘ above 40° C., or above 45° C., or above 50° C., or above 55° C., or above 60° C., or above 65° C., or higher), such as PDC from Z. palmae. The amino acid and nucleic acid sequences of PDC from Z. palmae are provided in FIG. 34.

Typically, the enzyme is more active, when expressed and folded at a given temperature, than an otherwise equivalent unmodified enzyme. By the enzyme being more active, we include the meaning that it retains at least one biological activity to a greater extent than does an otherwise equivalent unmodified enzyme, when the modified and unmodified enzymes are expressed and allowed to fold at a given temperature. Thus, the enzyme may be more active than an otherwise equivalent unmodified enzyme when expressed and folded at a temperature of at least 20° C., or at least 25° C., or at least 30° C., or at least 35° C., or at least 40° C. or at a temperature of at least 45° C., or at least 50° C., preferably at least 55° C., or at least 60° C., or at least 65° C., or at least 70° C., or at least 75° C., or at least 80° C., or at least 85° C., or at least 90° C.

Preferably, the expressed enzyme is at least 25%, at least 50% or at least 100% more active, than an otherwise equivalent, unmodified, enzyme, when expressed and allowed to fold at a temperature of at least 20° C., or at least 25° C., or at least 30° C., or at least 35° C., or at least 40° C., or at least 45° C., or at least 50° C., preferably at least 55° C. or at least 60° C. or at least 65° C., or at least 70° C., or at least 75° C., or at least 80° C., or at least 85° C., or at least 90° C. More preferably, the expressed enzyme is at least 2×, or at least 3× or at least 4× or at least 5× more active, than an otherwise equivalent, unmodified, enzyme, when expressed and folded at these temperatures. At temperatures of expression and folding above 55° C., the expressed enzyme may be at least 10×, or at least 20× more active, than an otherwise equivalent, unmodified, enzyme, at these temperatures.

Typically, the expressed enzyme is active when expressed and folded at a temperature of above 50° C., such as 51° C., 52° C., 53° C., 54° C., 55° C., 56° C., 57° C., 58° C., or 59° C., or yet more preferably at 60° C. or 65° C. or 70° C. or 75° C. or 80° C. or above, and retains at least one biological activity of an unmodified enzyme at these increased temperatures.

In a preferred embodiment, by the enzyme being active when expressed and folded at these higher temperatures, we include the meaning that it retains a detectable e.g. at least 1%, or at least 2%, or at least 3%, or at least 4%, or at least 5%, more preferably at least 10% of a biological activity of an otherwise equivalent unmodified enzyme which has been folded at 30° C. and whose activity is measured at 30° C., as described below.

Still more preferably, the enzyme can be expressed and folded at 50° C. or 55° C. or 60° C. or 65° C. or 70° C. or 75° C. or 80° C. or higher, and retains at least 20%, or at least 30%, or at least 40%, or at least 50%, or at least 60%, or at least 70% of a biological activity of an otherwise equivalent unmodified enzyme which has been folded at 30° C. and whose activity is measured at 30° C., as described below. It is further preferred that the enzyme can be expressed and folded at a higher temperature as described above, and preferably at 50° C. or 55° C. or 60° C. or 65° C. or 70° C. or 75° C. or 80° C. or higher, and retain at least 75%, or at least 80%, or at least 85%, or at least 90%, or at least 95%, or at least 100% or more of a biological activity of an otherwise equivalent unmodified enzyme which has been folded at 30° C. and whose activity is measured at 30° C., as described below.

Typically, the method improves the folding of an enzyme such that it can be expressed and folded at higher temperatures than an otherwise equivalent unmodified enzyme. Thus, the method may allow the enzyme to retain detectable e.g. 1%, or at least 2%, or at least 3%, or at least 4%, or at least 5%, more preferably at least 10% of a biological activity when expressed and folded at a temperature of at least 1° C. more than the highest temperature at which an otherwise equivalent unmodified enzyme can be expressed and folded, and retain detectable biological activity. This is conveniently referred to herein as an improvement in thermostability of folding of at least 1° C. More preferably, the method allows for an improvement in the thermostability of folding, relative to unmodified enzyme, of at least 2° C., or at least 3° C., or at least 4° C., or at least 5° C. Still more preferably, the method allows for an improvement in the thermostability of folding, relative to unmodified enzyme, of at least 6° C., or at least 7° C., or at least 8° C., or at least 9° C. Yet more preferably, the thermostability of folding is improved, relative to the unmodified enzyme, by at least 10° C., or at least 15° C., or at least 20° C., or more.

Suitably, this method allows an enzyme to be produced (e.g., expressed and correctly folded) in a functional form at the limits of its thermostability. For instance, the PDC from Zymomonas palmae is thermostable (by tests on an enzyme folded at 30-37° C.) up to 65° C. Thus, once made, this enzyme does not unfold significantly until 65° C. However, we have shown that it does not fold properly above 50-52° C. Thus, in the case of Z. palmae PDC, the thermostability of folding can be improved by about 13-15° C.; i.e., from about 50-52° C. up to about 65° C.

The biological activity may be any suitable biological activity that can be used as a readout as to whether the enzyme has folded correctly, since an improperly folded enzyme is inactive. Suitable biological activities may include enzymic activity, binding activity or a signalling-pathway modulation activity, but enzymic activities are preferred. Preferably, the biological activity measured to assess folding is the specific activity of the enzyme, i.e. the activity per unit amount of enzyme.

Conveniently, the biological activity of the modified enzyme is assessed by growing cells that express the enzyme at one or more temperatures. Extracts from the cells grown at one or more temperatures may then be tested for biological activity of the enzyme. The biological activity can then be compared to that of an otherwise, equivalent unmodified enzyme, expressed under the same conditions.

Binding activity may be assessed by measuring substrate or analogue binding using techniques well known in the art, including for example equilibrium dialysis or surface plasmon resonance. TPP binding may also be used as an indicator of correct folding since once the enzyme is folded the TPP is less strongly bound.

Preferably, the enzyme activity that is measured is a keto-acid decarboxylase activity, such as PDC activity. Suitable methods for measuring this activity are well known in the art and are described, for example in Hoppner & Doelle (1983). An example of how this may be achieved is set out in Example 4.

By folding of the enzyme comprising a TPP binding domain we include the meaning that the protein is formed into the correct secondary and tertiary structure that enables activity. Typically, this includes folding the protein monomer into the correct dimeric, tetrameric or multimeric forms.

It is appreciated that the conditions which allow the folding of the enzyme include the presence of TPP and Mg²⁺ ions, as shown in FIG. 25.

Although it is convenient to assess enzyme folding by using a biological activity as a read-out, other methods, including biophysical methods, are known in the art. For example, changes in fluorescence spectra can be a sensitive indicator of unfolding, either by use of intrinsic tryptophan fluorescence or the use of extrinsic fluorescent probes such as 1-anilino-8-napthaleneulfonate (ANS), for example as implemented in the Thermofluor™ method (Mezzasalma et al (2007). Proteolytic stability, deuterium/hydrogen exchange measured by mass spectrometry, blue native gels, capillary zone electrophoresis, circular dichroism (CD) spectra, NMR and light scattering may also be used to measure unfolding by loss of signals associated with secondary or tertiary structure. Such methods as applied to keto-acid decarboxylases are described in Pohl et al (1994).

It is appreciated that, typically, the one or more TPP binding domains are at the N-terminus and/or C-terminus of the enzyme comprising a TPP binding domain (e.g. keto-acid decarboxylase, such as PDC), as shown schematically in FIG. 16. TPP binding domains are highly conserved between proteins, and since proteins containing them are well known in the art, it has been possible to define a TPP binding enzyme family (see FIG. 18). Some TPP binding domains are shown in FIGS. 1-4, 19, 20 and 33.

It is preferred that the N-terminal TPP binding domains that are replaced include the N terminal alpha helix, which may be involved in initial capture of TPP, as well as the main TPP binding site. In other embodiments, however, only one of these two N-terminal regions is replaced with the equivalent region from a thermostable TPP binding domain. Thus, it will be appreciated that the N-terminal TPP binding domain includes both the N-terminal alpha helix and the main TPP binding site and either or both of these portions may be replaced.

It will also be understood that replacing one or more TPP binding domains at either the N-terminus and/or C-terminus may comprise replacing a part of the main TPP binding site, and in the case of the N-terminal TPP binding domain a part of the N-terminal alpha helix. For instance, as described in the Examples and in FIG. 34, replacing a TPP binding domain at the C-terminus may comprise replace a part of the main TPP binding site.

It is also appreciated that it is not only the TPP binding domains that may be replaced, but some surrounding sequence as well. Thus, in an embodiment, it is possible to modify the enzyme comprising the TPP binding domain by replacing the up to 100 amino acid residues (e.g. up to 15, 20, 25, 30, 35, 40, 45, 50, 55, 60, 65, 70, 75, 80, 85, 90 or 95 amino acids) at the N-terminus, and/or up to 150 amino acids residues at the C-terminus (e.g. up to 15, 20, 25, 30, 35, 40, 45, 50, 55, 60, 65, 70, 75, 80, 85, 90, 95, 100, 105, 110, 115, 120, 125, 130, 135, 140 or 145 amino acids), with the equivalent region from the thermostable TPP binding protein.

By replacing one or more TPP binding domains, we also include the meaning of replacing a consecutive amino acid sequence of up to 100 amino acids within a window defined by the 100 amino acids at the N-terminus (e.g. up to 15, 20, 25, 30, 35, 40, 45, 50, 55, 60, 65, 70, 75, 80, 85, 90 or 95 amino acids) and/or up to 150 amino acids within a window defined by the 150 amino acids at the C-terminus within the C-terminus (e.g. up to 15, 20, 25, 30, 35, 40, 45, 50, 55, 60, 65, 70, 75, 80, 85, 90, 95, 100, 105, 110, 115, 120, 125, 130, 135, 140 or 145 amino acids), with the equivalent region from the thermostable TPP binding protein. Thus, replacing one or more TPP binding domains may involve replacing stretches of consecutive amino acids at the N- and/or C-termini, or it may involve replacing stretches of consecutive amino acids near to the N- and/or C-termini but which do not include the terminal amino acids. In the case of replacing an N-terminal TPP binding domain, it is preferred if the replaced amino acids include at least a portion of the N-terminal alpha helix and/or at least a portion of the C-terminal TPP binding site. In the case of replacing a C-terminal TPP binding domain, it is preferred if the replaced amino acids include at least a portion of the C-terminal TPP binding site.

TPP binding domains in enzymes (e.g. keto-acid decarboxylases) especially pyruvate decarboxylases from many organisms are well known in the art and can readily be obtained from sequence contained in libraries such as GenBank, and in bioinformatic databases such as NCBI.

The TPP binding domain at the N-terminus of an enzyme comprising a TPP binding domain (e.g. a keto-acid decarboxylase such as pyruvate decarboxylase) may be an amino acid sequence that corresponds to the N-terminal TPP binding domain of the Z. palmae pyruvate decarboxylase (PDC), whose sequence is set out in FIG. 1. This sequence includes the TPP binding site (underlined) and the surrounding sequences such as the N-terminal alpha helix region.

The TPP binding domain at the C-terminus of an enzyme comprising a TPP binding domain (e.g. a keto-acid decarboxylase such as pyruvate decarboxylase) may be an amino acid sequence that corresponds to the C-terminal TPP binding domain of the Z. palmae PDC whose sequence is set out in FIG. 2. This sequence includes the TPP binding site (underlined) and the surrounding sequences.

By an amino acid sequence that corresponds to the N- or C-terminal TPP binding domain of the Z. palmae PDC we mean the region of an enzyme (e.g. a keto-acid decarboxylase such as PDC) from a different organism that aligns to the Z. palmae TPP binding domain, when the two sequences are compared using an alignment tool such as MacVector and CLUSTALW. It will be appreciated that TPP binding domains in further protein sequences can be identified accordingly. Also, it is possible to perform a BLAST search via NCBI which will identify the potential sites automatically, for example by Pfam.

In preferred embodiments of this aspect of the invention, the thermostable TPP binding protein, from which the TPP-binding domain is taken, may be derived from a thermophilic organism.

It may be preferred that the thermostable TPP-binding protein, from which the TPP-binding domain is taken, is an acetolactate synthase, which may suitably be from a Geobacillus sp., such as G. thermoglucosidasius or G. kaustophilus. Other enzymes from which thermostable TPP binding domains may be taken include any of the following, which are preferably derived from thermophiles: an alpha-ketoacid dehydrogenase, a branched chain amino acid dehydrogenase, a transketolase, a 2-hydroxyphytanoyl-CoA lyase, an alpha-ketoacid ferredoxin oxidoreductase, a glyoxylate carboligase, an alpha-ketoacid oxidase, a sulfoacetaldehyde acetyltransferase, a 2-succinyl-5-enolpyruvyl-6-hydroxy-3-cyclohexene-1-carboxylate synthase, a pyruvate synthase, an epi-inositol hydrolase, a pyruvate:flavodoxin oxidoreductase, a 3D-(3,5/4)-trihydroxycyclohexane-1,2-dione hydrolase, a 2-oxoglutarate synthase, a 2-succinyl-6-hydroxy-2,4-cyclohexadiene-1-carboxylic acid synthase, a phenylglyoxylate:acceptor oxidoreductase, and a myo-inositol catabolism protein lolD.

In an embodiment, the thermostable TPP binding domain is not taken from a keto-acid decarboxylase such as PDC.

Typically, the TPP binding domain of the thermostable TPP-binding protein is at the N-terminus or C-terminus of the thermostable TPP-binding protein.

In an embodiment, it is preferred that the TPP binding domains of the enzyme comprising a TPP binding domain (e.g. a keto-acid decarboxylase such as pyruvate decarboxylase) should be exchanged with TPP binding domains from the corresponding termini of the thermostable TPP-binding protein. Thus, for example, preferably, a ‘non-thermophilic’ N-terminal TPP binding domain is replaced by a ‘thermophilic’ N-terminal TPP binding domain, and a ‘non-thermophilic’ C-terminal TPP binding domain is replaced by a ‘thermophilic’ C-terminal TPP binding domain.

By replacing “one or more” TPP binding domains we mean that either one or both of the TPP binding domains in the monomeric form of the enzyme comprising a TPP binding domain (e.g. a keto-acid decarboxylase, such as PDC) has been replaced. In the active protein, however, which is in the form of a dimer, tetramer, or multi-dimer, an equivalent multiple of the one or both TPP binding domains is replaced. It is appreciated that for homo-dimers which comprise two identical subunits, replacing one or more TPP binding domains in the monomer will result in the equivalent replacement in the dimer.

When only one of the TPP binding domains in an enzyme comprising a TPP binding domain (e.g. a keto-acid decarboxylase such as pyruvate decarboxylase) is replaced, it may be the N-terminal TPP binding domain or the C-terminal TPP binding domain.

TPP binding domains in thermostable TPP binding proteins from many organisms are well known in the art. Generally, they correspond to the TPP binding domains in proteins from thermophiles that occupy the same relative position as the TPP binding proteins derived from mesophiles.

The TPP binding domain at the N-terminus of the thermostable TPP binding protein may be an amino acid sequence that corresponds to the N-terminal TPP binding domain of acetolactate synthase from G. kaustophilus whose sequence is set out in FIG. 3, or it may comprise or consist of this sequence.

The TPP binding domain at the C-terminus of the thermostable TPP binding protein may be an amino acid sequence that corresponds to the C-terminal TPP binding domain of acetolactate synthase from G. kaustophilus whose sequence is set out in FIG. 4, or it may comprise or consist of this sequence.

By an amino acid sequence that corresponds to the N- or C-terminal TPP binding domain of a thermostable TPP binding protein we mean the region of the thermostable TPP binding protein that aligns to the G. kaustophilus TPP binding domain, when the two sequences are compared using an alignment tool such as MacVector and CLUSTALW. It will be appreciated that thermostable TPP binding domains in further protein sequences can be identified accordingly. Also, it is possible to perform a BLAST search via NCBI which will identify the potential sites automatically, for example by Pfam. In this instance, it is preferred if the search is limited to proteins derived from thermophiles, for example using an appropriate search filter, as is commonly available such as in NCBI. Alternatively, a list of known homologues of TPP binding proteins (e.g. ALS) may be searched manually for proteins derived from known thermophiles (e.g. thermophilic bacteria).

In an embodiment, the TPP binding domains from the thermostable TPP binding proteins may be mutated to improve TPP binding, using methods, techniques and resources that are very well known in the art (Arnold & Volkov, 1999). Thus, in an embodiment, the method of the invention may include replacing one or more TPP binding regions in an enzyme comprising a TPP binding domain (e.g. a keto-acid decarboxylase such as pyruvate decarboxylase) with a TPP binding sequence that has at least 90% sequence identity to a TPP binding domain from a thermostable TPP binding protein, as described above. In this embodiment, it is more preferred that the TPP binding regions in the enzyme comprising a TPP binding domain (e.g. a keto-acid decarboxylase such as pyruvate decarboxylase) are replaced with TPP binding sequences that have at least 91%, or at least 92%, or at least 93%, or at least 94%, or at least 95%, or at least 96%, or at least 97%, or at least 98%, or at least 99% sequence identity to a TPP binding domain from a thermostable TPP binding protein. Thus, the TPP binding regions in the enzyme comprising a TPP binding domain (e.g. a keto-acid decarboxylase such as pyruvate decarboxylase) may be replaced with TPP binding sequences having 1 or 2 or 3 or 4 or 5 amino acid changes from the sequence of the TPP binding domain from a thermostable TPP binding protein. These 1 or 2 or 3 or 4 or 5 amino acid changes may, independently, be conservative or non-conservative, as is known in the art.

It is appreciated that the expression, and the folding, of the enzyme comprising a TPP binding domain (e.g. keto-acid decarboxylase such as pyruvate decarboxylase) may be carried out in a cell-free system in vitro. Alternatively, the expression, and the folding, of the enzyme comprising a TPP-binding domain (e.g. keto-acid decarboxylase such as pyruvate decarboxylase) may be carried out in vivo in a cellular system.

In one preferred embodiment, the enzyme comprising a TPP binding domain (e.g. keto-acid decarboxylase such as pyruvate decarboxylase) is expressed and folded in a thermophilic cell. By a thermophilic cell, we include the meaning of any cell (e.g. bacteria or archaea) which can grow optimally at temperatures above 50° C. Thus, it will be appreciated that by modifying an enzyme comprising a TPP binding domain according to the invention such as one derived from a mesophile, it is possible to express and fold that enzyme at higher temperatures characteristic of a thermophile's growing conditions.

The thermophilic cell may be a bacterial cell, for example a Gram positive or a Gram negative bacterial cell. The thermophilic cell may be a Geobacillus sp., such as G. thermoglucosidasius or G. kaustophilus. G. thermoglucosidasius may be preferred.

Conditions for growth and culture of thermophilic cells are well known in the art (Cripps et al (2009)).

It will be understood that the conditions that allow expression and folding of the enzyme may be optimised to further improve folding of the enzyme. For example, without wishing to be bound by any theory, the inventors believe that a high concentration of TPP, as achieved for example by adding thiamine to a culture medium, improves folding properties of enzymes.

In a specific embodiment, the nucleic acid encodes a Zymomonas, preferably a Z. palmae, PDC enzyme in which one or more TPP binding domains in the PDC enzyme are replaced with an N-terminal and/or C-terminal TPP binding domain of acetolactate synthase from G. kaustophilus, whose amino acid sequences are set out in FIGS. 3 and 4.

A second aspect of the invention provides a method of improving the thermostability of a enzyme comprising a TPP binding domain, the method comprising replacing one or more TPP binding domains in the enzyme comprising a TPP binding domain with a TPP binding domain from a thermostable TPP-binding protein.

Typically, the method improves thermostability of the enzyme comprising a TPP binding domain (e.g. keto-acid decarboxylase such as pyruvate decarboxylase) by at least 1° C. More preferably, the thermostability of the modified enzyme comprising a TPP binding domain (e.g. keto-acid decarboxylase such as pyruvate decarboxylase) is improved, relative to the unmodified enzyme, by at least 2° C., or at least 3° C., or at least 4° C., or at least 5° C. Still more preferably, the thermostability of the modified enzyme comprising a TPP binding domain (e.g. keto-acid decarboxylase such as pyruvate decarboxylase) is improved, relative to the unmodified enzyme, by at least 6° C., or at least 7° C., or at least 8° C., or at least 9° C. Yet more preferably, the thermostability of the modified enzyme comprising a TPP binding domain (e.g. keto-acid decarboxylase such as pyruvate decarboxylase) is improved, relative to the unmodified enzyme, by at least 10° C., or more.

The improved thermostability is typically measured in comparison to an unmodified enzyme comprising a TPP binding domain (e.g. keto-acid decarboxylase such as pyruvate decarboxylase), i.e., an otherwise equivalent enzyme in which the one or more TPP binding sites have not been replaced with TPP binding domains from a thermostable TPP-binding protein. Thermostability may be assessed used standard techniques known in the art. For example, thermostability is conveniently measured by an extended lifetime of the folded enzyme at a given temperature. Destabilisation under heat is typically determined by measuring denaturation or loss of structure. As is discussed herein, this may manifest itself by loss of a biological activity or loss of secondary or tertiary structure indicators.

Typically, improving the thermostability of the enzyme comprising a TPP binding domain (e.g. keto-acid decarboxylase such as pyruvate decarboxylase) allows it to be folded at a higher temperature, such as above 45° C., or above 50° C., and retain at least one biological activity of the unmodified enzyme. More preferably, improving the thermostability of the enzyme comprising a TPP binding domain (e.g. keto-acid decarboxylase such as pyruvate decarboxylase) allows it to be folded at above 51° C., 52° C., 53° C., 54° C., 55° C., 56° C., 57° C., 58° C., or above 59° C., or yet more preferably at above 60° C. or above 65° C., and retain at least one biological activity of the unmodified enzyme at these increased temperatures. It is thus appreciated that the folding of the protein can take place, and the activity can be measured, at 45° C., 46° C., 47° C., 48° C., 49° C., 50° C., 51° C., 52° C., 53° C., 54° C., preferably at 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C. or 85° C.

By the folded enzyme comprising a TPP binding domain (e.g. keto-acid decarboxylase such as pyruvate decarboxylase) retaining at least one biological activity of the unmodified enzyme we mean that it retains at least one biological activity of the equivalent unmodified enzyme at a detectable level.

More preferably, the thermostable enzyme comprising a TPP binding domain (e.g. keto-acid decarboxylase such as pyruvate decarboxylase) can be folded at a higher temperature as described above, and retain, at that temperature, at least 10% of a biological activity of an otherwise equivalent unmodified enzyme which has been folded at 30° C. and whose activity is measured at 30° C.

Still more preferably, the thermostable enzyme comprising a TPP binding domain (e.g. keto-acid decarboxylase such as pyruvate decarboxylase) can be folded at a higher temperature as described above, and retain, at that temperature, at least 20%, or at least 30%, or at least 40%, or at least 50%, or at least 60%, or at least 70% of a biological activity of an otherwise equivalent unmodified enzyme which has been folded at 30° C. and whose activity is measured at 30° C. It is further preferred that the thermostable enzyme comprising a TPP binding domain (e.g. keto-acid decarboxylase such as pyruvate decarboxylase) can be folded at the higher temperature as described above, and retains, at that temperature, at least 75%, or at least 80%, or at least 85%, or at least 90%, or at least 95%, or at least 100% or more of a biological activity of an otherwise equivalent unmodified enzyme which has been folded at 30° C. and whose activity is measured at 30° C.

In an embodiment, the biological activity may be an enzyme activity, a binding activity or a signaling pathway modulation activity, as described above in relation to the first aspect of the invention. It is preferred that the biological activity is an enzyme activity. Thus, typically, the biological activity is keto-acid decarboxylase activity, for example pyruvate decarboxylase activity. Methods for measuring keto-acid decarboxylase activity, including pyruvate decarboxylase activity, are well known in the art and are described above.

In one preferred embodiment, the thermostability of the enzyme comprising a TPP binding domain (e.g. keto-acid decarboxylase such as pyruvate decarboxylase) is improved when the enzyme is expressed in a thermophilic cell, including those described above. Thus, the thermostability of the enzyme may be improved when expressed in a Geobacillus sp. cell, such as G. thermoglucosidasius or G. kaustophilus. G. thermoglucosidasius.

The one or more TPP binding domains in the keto-acid decarboxylase enzyme may be replaced with a TPP binding domain from a thermostable TPP-binding protein, in any suitable way. Conveniently, modified keto-acid decarboxylase enzyme is encoded by a suitable nucleic acid molecule and expressed in a suitable host cell. Suitable nucleic acid molecules encoding the modified keto-acid decarboxylase enzyme may be made using standard cloning techniques, site-directed mutagenesis and PCR as is well known in the art.

In one preferred embodiment, the thermostability of the enzyme comprising a TPP binding domain is improved when the enzyme is expressed in a thermophilic cell, including those described above.

Details and preferences for the enzyme comprising a TPP binding domain are as defined above in the first aspect of the invention. It may be preferred that the enzyme is a keto-acid decarboxylase such as a pyruvate decarboxylase

Details and preferences for replacing one or more TPP binding domains in the enzyme comprising a TPP binding domain with a TPP binding domain from a thermostable TPP-binding protein are as defined above in the first aspect of the invention. Suitable recombinant methods for manipulating nucleic acid molecules are very well known in the art.

Details and preferences for the one or more TPP binding domains in the enzyme comprising a TPP binding domain are as defined above in the first aspect of the invention.

Details and preferences for the TPP binding domain from a thermostable TPP-binding protein are as defined above in the first aspect of the invention.

In a preferred embodiment of this aspect of the invention, the method may comprise replacing one or more TPP binding domains in a Zymomonas, preferably Z. palmae, PDC enzyme with an N-terminal and/or C-terminal TPP binding domain of acetolactate synthase from G. kaustophilus, whose amino acid sequences are set out in FIGS. 3 and 4.

A third aspect of the invention provides a modified, i.e., mutant, enzyme comprising a TPP binding domain (e.g., a keto-acid decarboxylase, such as a PDC) in which, compared to the equivalent unmodified enzyme comprising a TPP binding domain (e.g., an unmodified keto-acid decarboxylase, such as a PDC), one or more TPP binding domains have been replaced with a TPP binding domain from a thermostable TPP-binding protein.

Details and preferences for the enzyme comprising a TPP binding domain (e.g., a keto-acid decarboxylase, such as a PDC) are as defined above in the first aspect of the invention. It may be preferred that the keto-acid decarboxylase is a pyruvate decarboxylase

Details and preferences for replacing one or more TPP binding domains in the enzyme comprising a TPP binding domain (e.g., a keto-acid decarboxylase, such as a PDC) with a TPP binding domain from a thermostable TPP-binding protein are as defined above in the first aspect of the invention. Suitable recombinant methods for manipulating nucleic acid molecules are very well known in the art.

Details and preferences for the one or more TPP binding domains in the enzyme comprising a TPP binding domain (e.g., a keto-acid decarboxylase, such as a PDC) are as defined above in the first aspect of the invention.

Details and preferences for the TPP binding domain from a thermostable TPP-binding protein are as defined above in the first aspect of the invention.

In a specific embodiment, the modified enzyme comprising a TPP binding domain (e.g., a keto-acid decarboxylase, such as a PDC) may comprise an amino acid sequence set out in any of FIGS. 5-9.

Typically, the mutant enzyme comprising a TPP binding domain (e.g., a keto-acid decarboxylase, such as a PDC) is capable of being folded, and retaining activity, at a temperature above 35° C., or above 40° C. or above 45° C. or above 50° C. For example, the mutant enzyme comprising a TPP binding domain (e.g., a keto-acid decarboxylase, such as a PDC) is active when folded at a temperature of above 50° C., such as 51° C., 52° C., 53° C., 54° C., 55° C., 56° C., 57° C., 58° C., or 59° C., or yet more preferably at 60° C., or 65° C. or above. Details and preferences for the biological activity of the modified enzyme, and methods for determining the activity, are as defined above in the first and second aspects of the invention.

A fourth aspect of the invention provides a pyruvate decarboxylase enzyme that is capable of being folded at a temperature above 50-52° C., and which retains pyruvate decarboxylase activity at a temperature above 50-52° C.

In various embodiments, the pyruvate decarboxylase enzyme is capable of being folded, and retaining activity, at 53° C., 54° C., 55° C., 56° C., 57° C., 58° C., or 59° C., or yet more preferably at 60° C., or 65° C. or above. Methods for determining pyruvate decarboxylase activity are very well known in the art and are as defined above in the first aspect of the invention.

Typically, the pyruvate decarboxylase will exhibit a specific activity of at least 100 nmol/min/mg, and preferably upto 1-5 micromol/min/mg.

A fifth aspect of the invention provides a nucleic acid molecule encoding a enzyme comprising a TPP binding domain (e.g., a keto-acid decarboxylase, such as a PDC) according to the third aspect of the invention or a pyruvate decarboxylase according to the fourth aspect of the invention.

In a specific embodiment, the encoded enzyme may comprise or consist of the sequences in FIGS. 5-9, which may be encoded by nucleic acid molecules comprising or consisting of the sequences in FIGS. 10-14.

The nucleic acid molecule may be DNA or RNA, and is preferably DNA. It may comprise deoxyribonucleotides, ribonucleotides, modified nucleotides or bases, and/or their analogues, or any substrate that can be incorporated into a polymer by DNA or RNA polymerase, or by a synthetic reaction. The nucleic acid molecule can be single-stranded or double-stranded, but generally is double-stranded DNA. The nucleic acid may be one that is free of sequences which naturally flank the nucleic acid molecule (i.e. sequences located at the 5′ and 3′ ends of the nucleic acid molecule) in the chromosomal DNA of the organism from which the nucleic acid is derived. A polynucleotide may comprise modified nucleotides, such as methylated nucleotides and their analogues. If present, modification to the nucleotide structure may be imparted before or after assembly of the polymer. The sequence of nucleotides may be interrupted by non-nucleotide components. Some specific examples of nucleic acid molecules encoding keto-acid decarboxylases of the invention are provided in FIGS. 10-14. Other suitable sequences can readily be determined for a given keto-acid decarboxylase based upon knowledge of the TPP-binding domains and the genetic code.

The nucleic acid molecule of the invention may be produced using standard molecule biology techniques, including PCR, and may make use of the sequence information provided herein. Molecular biological methods for cloning and engineering genes and cDNAs, for mutating DNA, and for expressing polypeptides from polynucleotides in host cells are well known in the art, as exemplified in “Molecular cloning, a laboratory manual”, third edition, Sambrook, J. & Russell, D. W. (eds), Cold Spring Harbor Laboratory Press, Cold Spring Harbor, N.Y., incorporated herein by reference.

It is appreciated that the nucleic acid encoding the enzyme comprising a TPP binding domain (e.g., a keto-acid decarboxylase, such as a PDC), may, in addition to modifications to one or more TPP binding domains, comprises further modifications to confer one or more desirable traits on the enzyme, such as increase in expression, or an increase in conformational stability or an improvement in the enzyme's performance in a particular reaction pathway (e.g. production of ethanol or acetaldehyde as described in more detail below). Such traits may be selected for using standard mutagenesis technology and directed evolution that is very well known in the art (see FIG. 32).

A sixth aspect of the invention provides a vector comprising the nucleic acid molecule according to the fifth aspect of the invention.

The vector can be of any type, for example a recombinant vector such as an expression vector. The expression vectors contain elements (e.g., promoter, signals of initiation and termination of translation, as well as appropriate regions of regulation of transcription) which allow the expression and/or the secretion of the enzymes comprising a TPP binding domain (e.g., a keto-acid decarboxylase, such as a PDC) in a host cell. Any of a variety of host cells can be used, such as a prokaryotic cell, for example, E. coli, or a eukaryotic cell, for example a mammalian cell, or a yeast, insect or plant cell. Many suitable vectors and host cells are very well known in the art. E. coli vectors such as pUC18 and E. coli-Geobacillus shuttle vectors such as pUCG18 are particularly preferred.

A seventh aspect of the invention provides a host cell comprising a nucleic acid molecule according to the fifth aspect of the invention or a vector according to the sixth aspect of the invention.

The host cell is typically a bacterial cell. For manipulation of the nucleic acid molecule, the cell may be E. coli or other common laboratory line.

For expression of the encoded enzyme, the host cell is preferably a thermophilic cell, such as one defined above with respect to the first aspect of the invention. Details and preferences for suitable thermophile cells are given above with respect to the first aspect of the invention. It may be preferred to use the cell that is the subject of Cripps et al (2009), incorporated herein by reference. It may be preferred to engineer the cell as a lactate dehydrogenase negative mutant of DL33, which we call DL44.

Typically, the host cell is a recombinant host cell and the nucleic acid encoding the decarboxylase enzyme is a heterologous nucleic acid. By a ‘heterologous nucleic acid’ we include the meaning of a nucleic acid that is not native to the genome of a particular host cell.

It will be appreciated that the host cell may be one that has already been selected or engineered to have certain desirable properties and suitable for further modification according to the invention, as described further below.

In a preferred embodiment, the nucleic acid molecule encoding the enzyme comprising a TPP binding domain (e.g., a keto-acid decarboxylase, such as a PDC) is one that has been selected for improved codon usage in the host cell or acellular extract thereof. By todon usage′ we include the meaning of analysing a given nucleic acid molecule being considered for expression in a recipient host cell (or acellular extract thereof) for the occurrence or “usage” of certain codons that the host cell will require (advantageously at sufficient levels) in order to translate the nucleic acid into a corresponding polypeptide. Based on such observations the recipient host cell may be recombinantly supplemented with any necessary codon. Alternatively, another host can be selected with superior codon usage or the nucleic acid can be altered to no longer comprise a limiting codon (e.g., by introducing a silent mutation (s)).

In a preferred embodiment, when the enzyme is pyruvate decarboxylase, the host cell comprises a nucleic acid encoding a further polypeptide involved in ethanologenesis.

By a polypeptide involved in ethanologenesis we include the meaning of any polypeptide capable of conferring on a cell ethanologenic properties or capable of improving any aspect of cellular ethanologenesis such as, for example, substrate uptake, substrate processing, ethanol tolerance etc.

It is further preferable that the host cell is able to naturally transport and metabolise the major pentose monomers found in hemicelluloses and/or use cellubiose and short chain xylans as substrates. This can readily be assessed by growing the cells on these substrates as a sole carbon source, and if necessary measuring their consumption by an appropriate technique such as HPLC.

More preferably, the host cell further comprises a nucleic acid encoding alcohol dehydrogenase. As is well known in the art, and discussed above, pyruvate decarboxylase and alcohol dehydrogenase, are two central enzymes in the ethanol production pathway. By the term “alcohol dehydrogenase” we include the meaning of any enzyme capable of converting acetaldehyde into an alcohol such as ethanol.

It is thus preferred that the host cell is ethanologenic, i.e., the host cell has the ability to produce ethanol from a carbohydrate as a primary fermentation product. This includes naturally occurring ethanologenic host cells, ethanologenic host cells with naturally occurring or induced mutations, and host cells which have been genetically modified to become ethanologenic.

In an embodiment, the host cell is suitable for fermenting ethanol from a sugar, such as fermenting ethanol as the primary product of fermentation.

In an embodiment, the host cell is suitable for fermenting ethanol from lignocellulosic feedstock, such as hemicellulose, cellobiose, or short chain xylans.

An eighth aspect of the invention provides a method for producing acetaldehyde comprising culturing the host cell of the seventh aspect of the invention, wherein the enzyme is pyruvate decarboxylase, under conditions effective to produce acetaldehyde.

The method may further comprise isolating and/or purifying the acetaldehyde, using methods very well known in the art.

A ninth aspect of the invention provides a method for producing ethanol comprising culturing the host cell of the seventh aspect of the invention, wherein the enzyme is pyruvate decarboxylase, under conditions effective to produce ethanol. As described above, the host cell capable of producing ethanol should also express alcohol dehydrogenase

The method may further comprise isolating and/or purifying the ethanol, using methods very well known in the art.

Preferably, the host cell is a Geobacillus (or other thermophile capable of fermentation) in which the natural fermentation pathways have been activated. Conveniently, the cells would be grown anaerobically or under conditions designed to force the cells to grow by fermentation as is standard practice in the art.

Typically, in the eighth and ninth aspects of the invention, the host cell is cultured at a temperature of at least 50° C. In various embodiments, the host cell is cultured at 51° C., 52° C., 53° C., 54° C., 55° C., 56° C., 57° C., 58° C., or 59° C., or yet more preferably at 60° C., or 60-65° C., or above.

In the eighth and ninth aspects of the invention, it may be preferred that the substrate in the culture medium is derived from a lignocellulosic feedstock such as any one or more of hemicellulose, cellobiose and short-chain xylans. Other substrates include starch (e.g. from grain) or sucrose (e.g. from beet and/or cane).

It is appreciated that the lignocellulosic feedstock may be subjected to enzyme hydrolysis at a temperature of at least 50° C. and the hydrolysate may be transferred directly into a vessel comprising a culture of the host cell at a temperature of at least 50° C.

A specific embodiment of the ninth aspect of the invention provides a method of producing ethanol comprising culturing a G thermoglucosidasius cell that comprises a nucleic acid encoding a Zymomonas, preferably Z. palmae, pyruvate decarboxylase, in which one or more thiamine pyrophosphate (TPP) binding domains in the pyruvate decarboxylase are replaced with a TPP binding domain from a thermostable TPP-binding protein, under conditions effective to produce ethanol, at a temperature of at least 50° C. More preferably, the temperature may be at least 55° C.

Optimally, the method is carried out on an industrial scale, preferably in continuous culture.

It will be appreciated that the host cell of the seventh aspect of the invention may be used in methods to produce other desired products. For example, by using an appropriate decarboxylase, it may be possible to produce isobutanol or isopentanol. Thus, the invention also includes a method for producing isobutanol or isopentanol, comprising culturing the host cell of the seventh aspect of the invention, wherein the enzyme comprising a TPP binding domain is a decarboxylase, under conditions effective to produce isobutanol or isopentanol. Preferences for the host cell are as described herein.

A tenth aspect of the invention provides a culture medium comprising acetaldehyde or ethanol produced according to the eighth and ninth aspects of the invention.

An eleventh aspect of the invention provides acetaldehyde or ethanol obtainable or obtained (e.g., produced, isolated and/or purified) by the methods of the eighth or ninth aspects of the invention.

A twelfth aspect of the invention provides an enzyme extract comprising detectable levels of the enzyme comprising a TPP binding domain (e.g. keto-acid decarboxylase activity such as pyruvate decarboxylase activity) derived from the host cell of the seventh aspect of the invention.

A thirteenth aspect of the invention provides a method of selecting a enzyme comprising a TPP binding domain (e.g., a keto-acid decarboxylase, such as a PDC) that can fold and retain enzyme activity above a particular temperature, comprising:
replacing one or more thiamine pyrophosphate (TPP) binding domains in the enzyme comprising a TPP binding domain (e.g., a keto-acid decarboxylase, such as a PDC) monomer with a TPP binding domain from a thermostable TPP-binding protein, or a variant thereof, and
assessing whether the modified enzyme can fold and retain enzyme activity above the particular temperature.

Typically, the particular temperature is 50° C. However, the particular temperature at which the mutant enzyme comprising a TPP binding domain (e.g., a keto-acid decarboxylase, such as a PDC) is capable of being folded, and retaining activity, may be 45-50° C., or a temperature above 50° C., such as 51° C., 52° C., 53° C., 54° C., 55° C., 56° C., 57° C., 58° C., or 59° C., 60° C., or 60-65° C. Details and preferences for the biological activity of the modified enzyme, and methods for determining the activity, are as defined above in the first and second aspects of the invention.

Details and preferences for the enzyme comprising a TPP binding domain (e.g., a keto-acid decarboxylase, such as a PDC) are as defined above in the first aspect of the invention. It may be preferred that the enzyme comprising a TPP binding domain (e.g., a keto-acid decarboxylase, such as a PDC) is a pyruvate decarboxylase.

Details and preferences for replacing one or more TPP binding domains in the enzyme comprising a TPP binding domain (e.g., a keto-acid decarboxylase, such as a PDC) with a TPP binding domain from a thermostable TPP-binding protein are as defined above in the first aspect of the invention.

Details and preferences for the one or more TPP binding domains in the enzyme comprising a TPP binding domain (e.g., a keto-acid decarboxylase, such as a PDC) are as defined above in the first aspect of the invention.

Details and preferences for the TPP binding domain from a thermostable TPP-binding protein are as defined above in the first aspect of the invention. By variants of a TPP binding domain from a thermostable TPP binding protein we include sequences that have at least 90% sequence identity to a TPP binding domain from a thermostable TPP binding protein, as described above. In this embodiment, the TPP binding regions in the enzyme (e.g., a keto-acid carboxylase, such as PDC) may be replaced with TPP binding sequences that have at least 91%, or at least 92%, or at least 93%, or at least 94%, or at least 95%, or at least 96%, or at least 97%, or at least 98%, or at least 99% sequence identity to a TPP binding domain from a thermostable TPP binding protein. Thus, the TPP binding regions in the enzyme comprising a TPP binding domain may be replaced with TPP binding sequences having 1 or 2 or 3 or 4 or 5 amino acid changes from the sequence of the TPP binding domain from a thermostable TPP binding protein. These 1 or 2 or 3 or 4 or 5 amino acid changes may, independently, be conservative or non-conservative, as is known in the art. Methods and techniques for random or directed mutation within a specific region of a protein are very well known in the art.

The invention will now be described with the aid of the following figures and examples.

### FIGURES

FIG. 1: Amino acid sequence of N-terminal TPP binding domain in Z. palmae pyruvate decarboxylase.

FIG. 2: Amino acid sequence of C-terminal TPP binding domain in Z. palmae pyruvate decarboxylase.

FIG. 3: Amino acid sequence of N-terminus TPP binding domain of the acetolactate synthase G. kaustophilus.

FIG. 4: Amino acid sequence of C-terminus TPP binding domain of the acetolactate synthase G. kaustophilus.

FIG. 5: Amino acid sequence of hybrid pyruvate decarboxylase N₁.

FIG. 6: Amino acid sequence of hybrid pyruvate decarboxylase N₂.

FIG. 7: Amino acid sequence of hybrid pyruvate decarboxylase N₁C.

FIG. 8: Amino acid sequence of hybrid pyruvate decarboxylase N₂C.

FIG. 9: Amino acid sequence of hybrid pyruvate decarboxylase C.

FIG. 10: Nucleotide sequence of hybrid pyruvate decarboxylase N₁.

FIG. 11: Nucleotide sequence of hybrid pyruvate decarboxylase N₂.

FIG. 12: Nucleotide sequence of hybrid pyruvate decarboxylase N₁C.

FIG. 13: Nucleotide sequence of hybrid pyruvate decarboxylase N₂C.

FIG. 14: Nucleotide sequence of hybrid pyruvate decarboxylase C.

FIG. 15: Prior art. This figure shows the thermostability of PDC enzymes. Recombinant ZmoPDC (•), ZpaPDC (▪), ApaPDC (∘), and SvePDC (□) proteins were preincubated at the temperatures indicated in 50 mM sodium citrate buffer at pH 5.0 with 1 mM TPP and 1 mM MgCl₂ for 30 min, cooled to 0° C., and assayed for residual activity at 25° C. in the same buffer (taken from Raj et al, 2002).

FIG. 16: Orientation of TPP binding sites in 2 monomers of PDC.

FIG. 17: (A) Coupled NADH assay. The conversion of pyruvate into acetaldehyde by the PDC is equimolar with the conversion of NADH into NAD+, and it can be measured by disappearance of NADH at 340 nm. (B). Lactate dehydrogenase reaction. Ref: http://biochem.co/page/2/. Pyruvate is converted into lactate while a molecule of NADH is oxidised to NAD+.

FIG. 18: Extract from NCBI showing that TPP binding sites are well established and enables the definition of a TPP enzyme binding family.

FIG. 19: Structural comparison of the N-termini of Z. palmae PDC and G. kaustophilus ALS. Alignment of the 2D structure predictions for the G. kaustophilus ALS against Z. palmae PDC, showing the degree of residue-specific similarity. Highlighted regions represent alpha helix and beta sheet and the TPP-binding site is underlined.

FIG. 20: Partial sequence alignment of published ZpPDC accession number: AAM49566.1 and PDC hybrids. TPP binding sites are underlined. In blue: N-terminal from GkALs (G. kaustophilus ALS); In green: N-terminal TPP binding site from GkALS. In yellow: C-terminal TPP binding site from GkALs. ClustalW2-EBI: (*) Identical (:) conserved substitution (.) semi-conserved substitution ( )non-conserved substitution.

FIG. 21: Fermentation pathways in G. thermoglucosidasius DL33, DL62 and DL81. X indicates enzymic steps that are knocked-out to improve ethanologenicity.

FIG. 22: Fermentation pathways in G. thermoglucosidasius DL62 for improved production of ethanol.

FIG. 23: Expression of PDC_{Zm} in G. thermoglucosidasius. This shows the distinction between standard thermostability and thermostability of folding.

FIG. 24: Pyruvate decarboxylase structure.

FIG. 25: Folded protein thermostability vs thermostability of folding.

FIG. 26: Expression and specific activity of C-terminal hybrid PDC in E. coli.

FIG. 27: Expression of C-terminal hybrid PDC in E. coli. Effects of thiamine concentration during growth, and temperature on fraction of original lysate of PDC present, are shown.

FIG. 28: PDC activity of C terminal hybrid expressed in G. thermoglucosidasius DL62.

FIG. 29: Expression of wild type PDC and H1 PDC hybrid.

FIG. 30: Expression of PDCs: Left hand panel—expression of H4 hybrid PDC; Right hand panel—expression of wild type PDC and hybrid PDCs H1, H2, H3 and H4.

FIG. 31: Specific activity of wild type Z. palmae PDC, and N and C terminal hybrid PDC, H4, following expression at different temperatures.

FIG. 32: Hybrid PDC and directed evolution. Schematic depiction of directed evolution method to select for PDC with desirable functional traits, e.g. ability to fold at high temperatures.

FIG. 33: Protein sequence of four PDC hybrids designed. N-terminal regions originating from the G. kaustophilus ALS are boxed. Non-boxed sequence represents the adjoining Z. palmae PDS sequence up to approximately residue 65. The TPP-binding site is underlined. Hybrids 3 and 4 differ from Hybrids 1 and 2 respectively in the addition of a C-terminal modification previously achieved.

FIG. 34: (A) Amino acid sequence and (B) nucleic acid sequence of Z. palmae PDC.

FIG. 35: N-terminal and C-terminal modifications of various PDC hybrids.

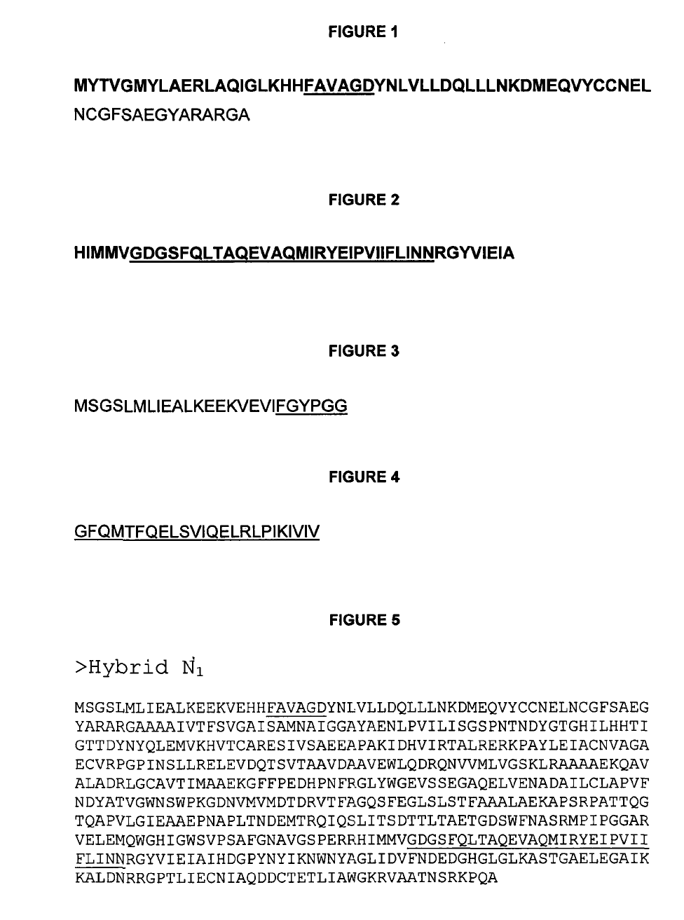

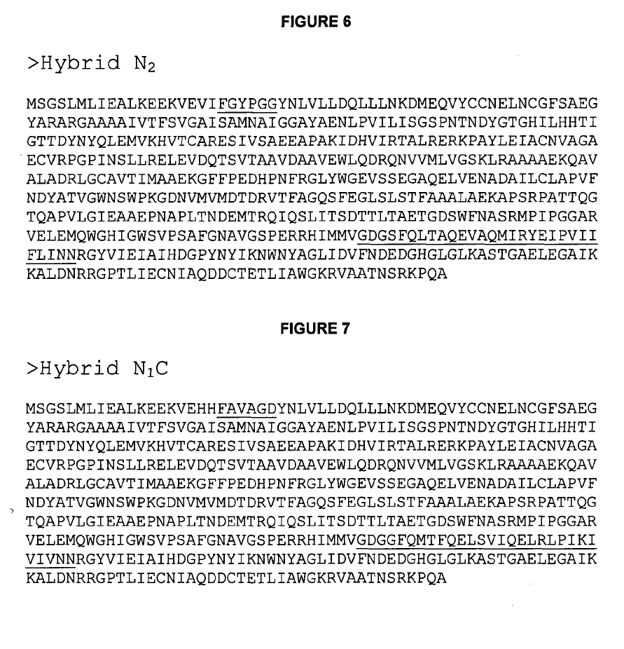

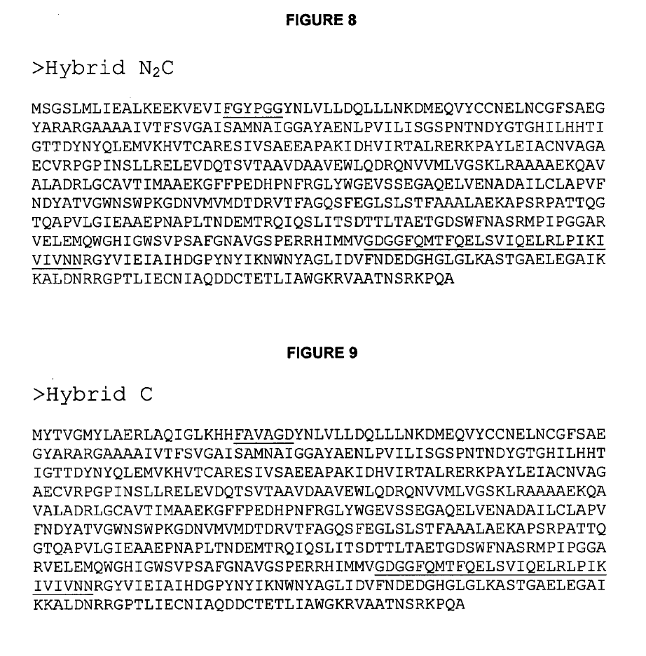

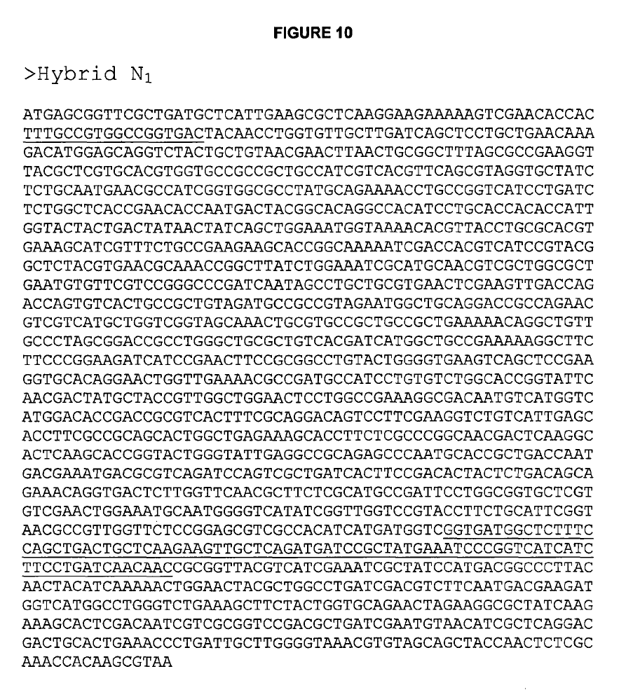

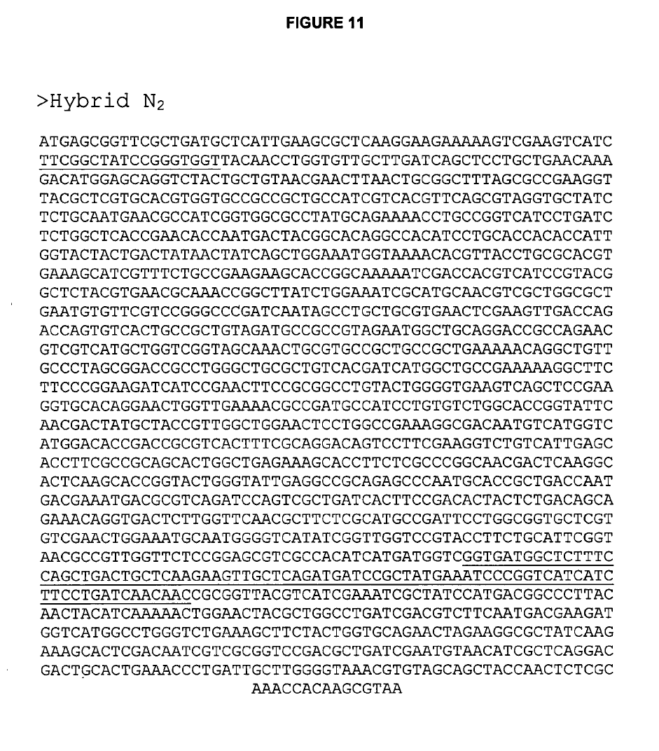

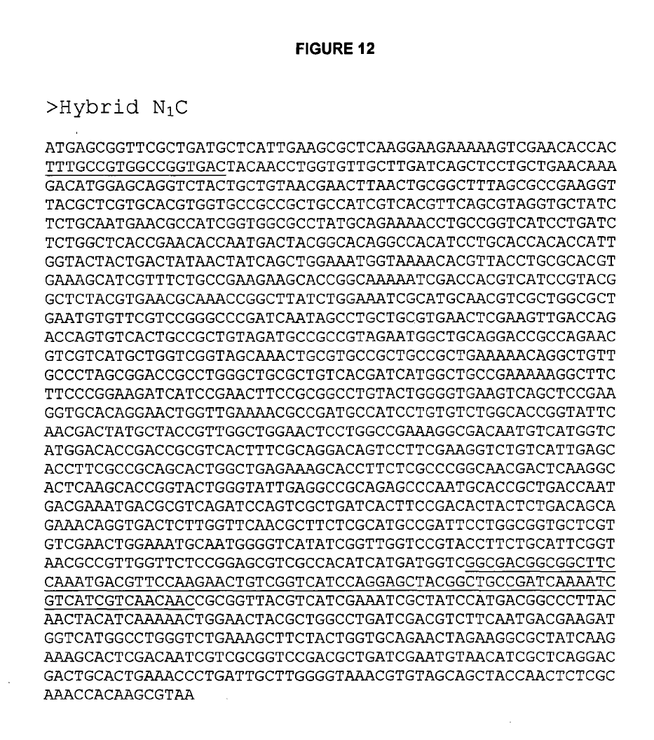

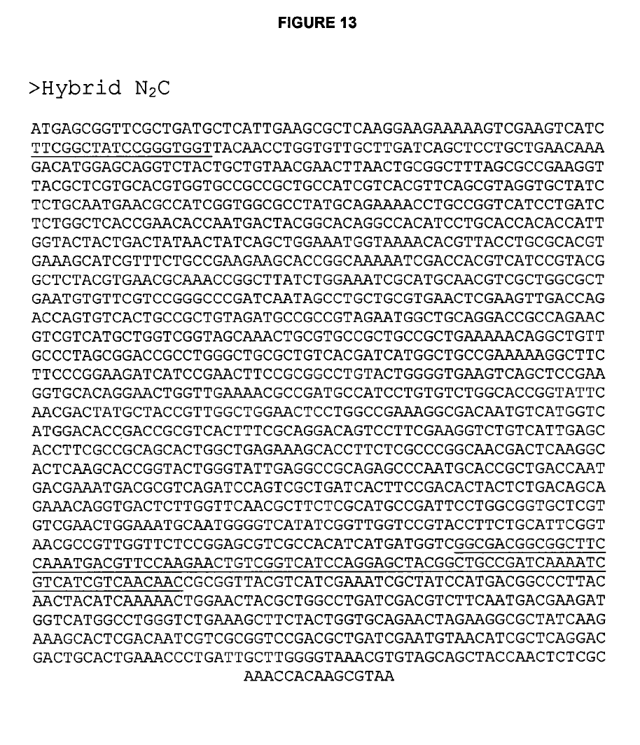

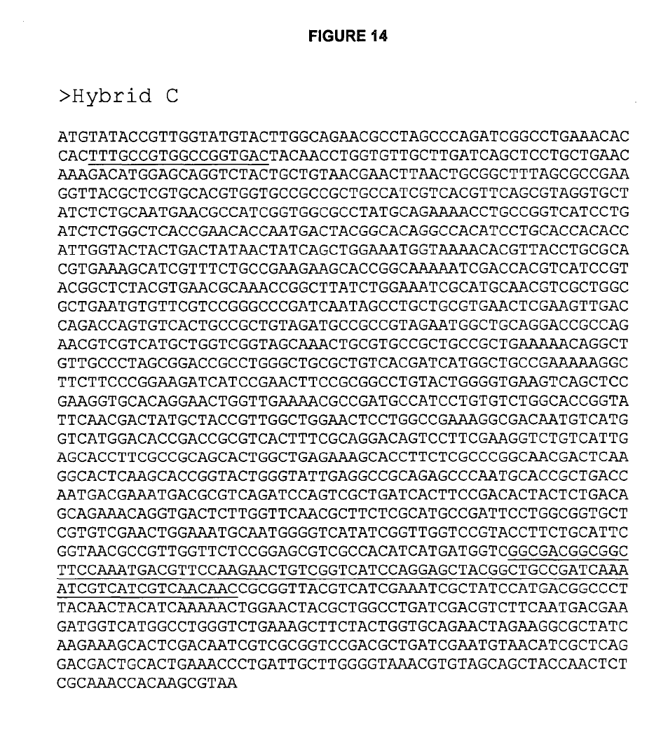

### EXAMPLES

### Example 1

### Activity of Z mobilis/palmae PDC when expressed in Geobacillus thermoglucosidasius

### Experiments and Results

We have shown that when PDC from Z. mobilis or Z. palmae is expressed in the thermophile Geobacillus thermoglucosidasius, PDC activity cannot be detected when cells are grown above 50-52° C., and even at this temperature, PDC activity was poor when compared to lower temperatures.

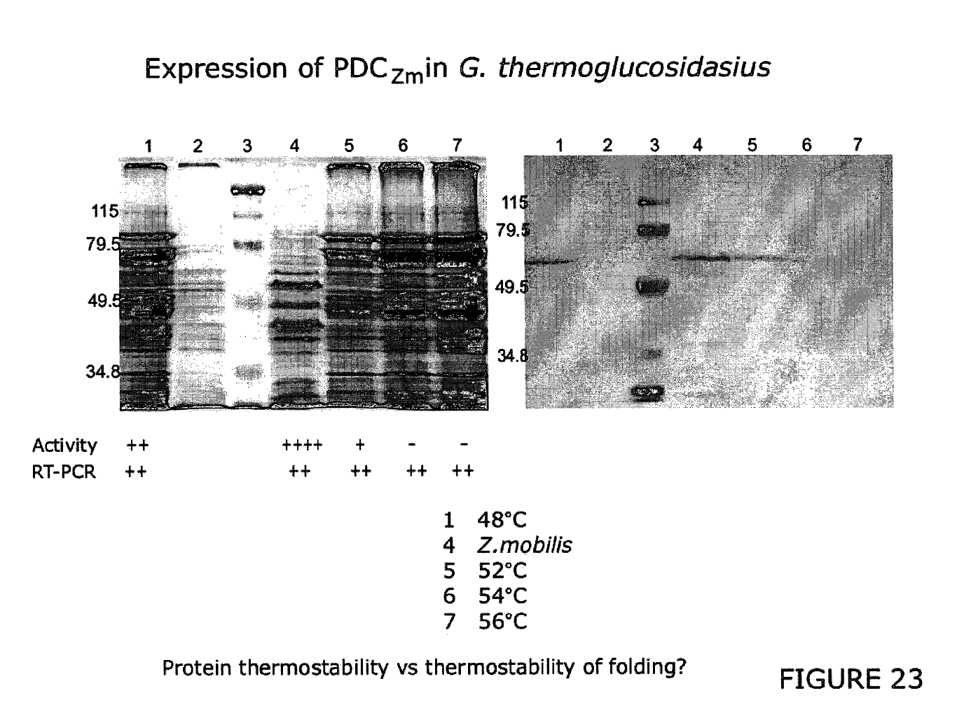

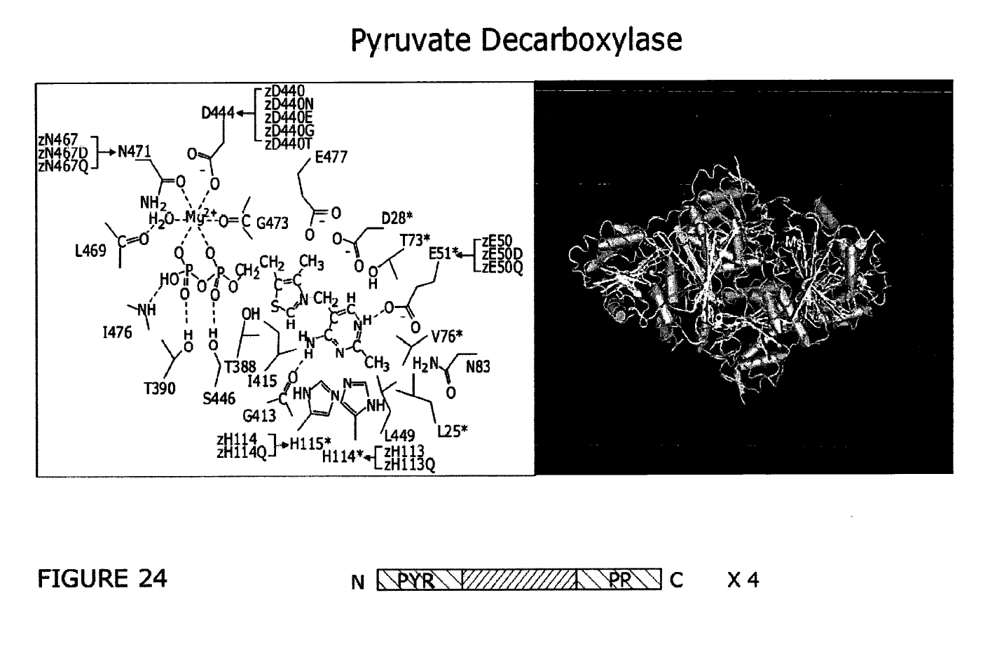

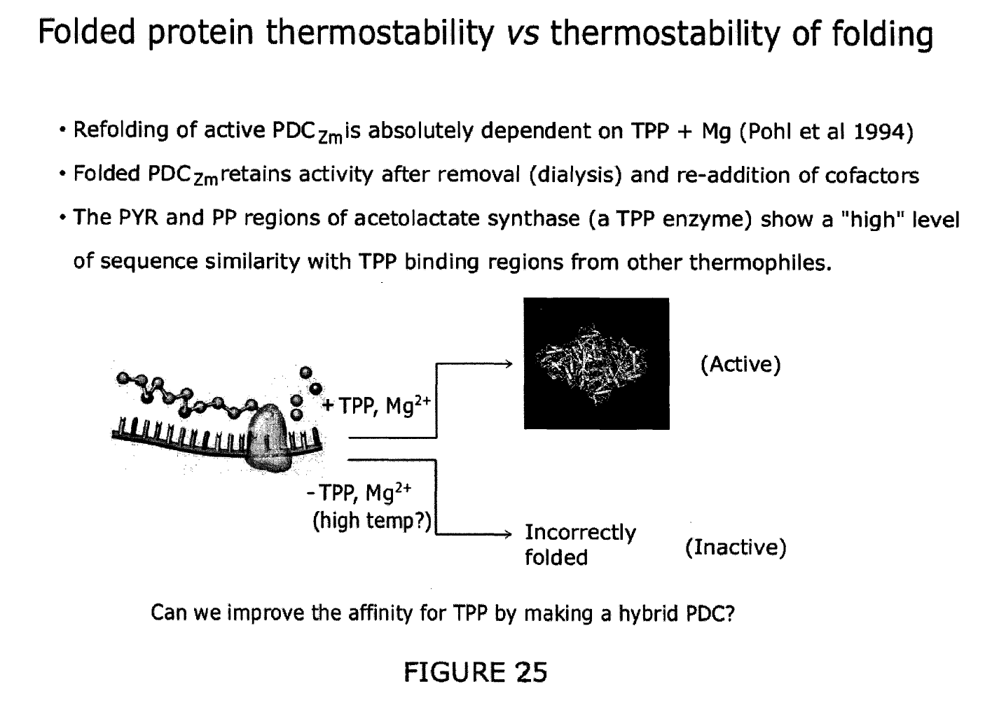

FIG. 23 shows mRNA and protein expression of PDC from Z. mobilis in G. thermoglucosidasius. Although still expressed, PDC activity decreases as temperature of expression increases.

### Discussion

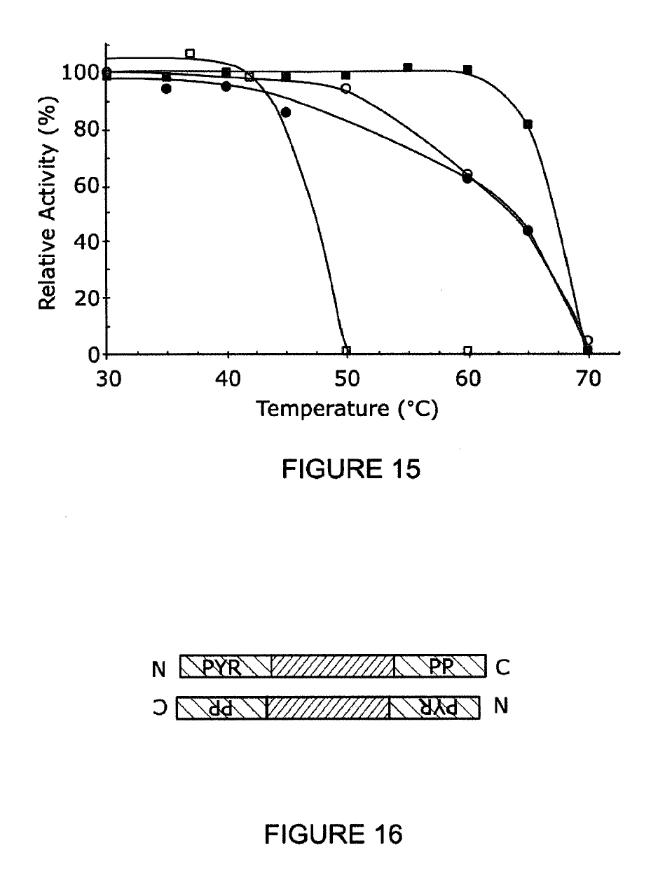

We were intrigued by these findings because the Z. palmae PDC in particular is known to be thermostable, i.e., once it has been synthesized in an active form in Z. palmae or E. coli at a low temperature, it can then been heated up to temperatures of 60-65° C., cooled down and retains activity in a standard assay (FIG. 15).

The difference between our results and those, for example, shown in FIG. 15 is that when the enzyme is produced in a thermophile, the protein has to fold correctly at high temperatures, whereas standard thermostability assays merely test the stability of the already folded protein.

Thus, we appreciated that Z. palmae PDC is unable to fold correctly at higher temperatures, which results in a loss of activity. The inability to fold correctly could be due to a number of factors.

In 1994, Pohl et al described that you could chemically denature the Z. mobilis PDC and it would refold into an active conformation if the cofactor thiamine pyrophosphate (TPP) was present, but it refolded into an inactive conformation when TPP was absent. Once PDC was in an inactive, addition of TPP did not restore activity.

We therefore hypothesised that the monomers of the PDC protein have to fold around the cofactor TPP in order to achieve the correct conformation. Under this hypothesis, TPP must bind to unfolded protein, the affinity of which will be temperature dependent. Thus, a possible reason why PDC does not fold correctly at high temperatures is that the TPP does not bind strongly enough to PDC, and an inactive conformation is formed.

TPP containing proteins, such as PDC, are dimers or multimers of dimers. The reason for this is that TPP binds between to N terminus of one subunit and the C terminus of the second (FIG. 16).

Some thermophiles do have TPP-containing proteins, and these proteins also have TPP binding sites at the N and C termini. We predicted that replacing the native TPP binding sites in the PDC (particularly at the N terminus) with TPP binding sites from a thermophilic TPP binding protein should allow TPP to bind to the modified PDC sufficiently strongly at high temperatures to allow the protein to fold around it. Accordingly, we believed that it might be possible to create a thermophilic PDC by changing the TPP binding sites of PDC to those from a more thermophilic protein. This is described in Example 2.

### Example 2

### Producing a Modified PDC Enzyme

### Experiments, Results and Discussion

We took the TPP binding site sequences from the protein acetolactate synthase (ALS) from G. kaustophilus as a guide and made hybrids in which regions of the N terminus and C terminus of the Z. palmae PDC were substituted with those from ALS. Using 2 different lengths of N terminal sequence (N₁ and N₂) and one modified C terminal sequence, five hybrids have been made in total (N₁, N₂, N₁C, N₂C, C).

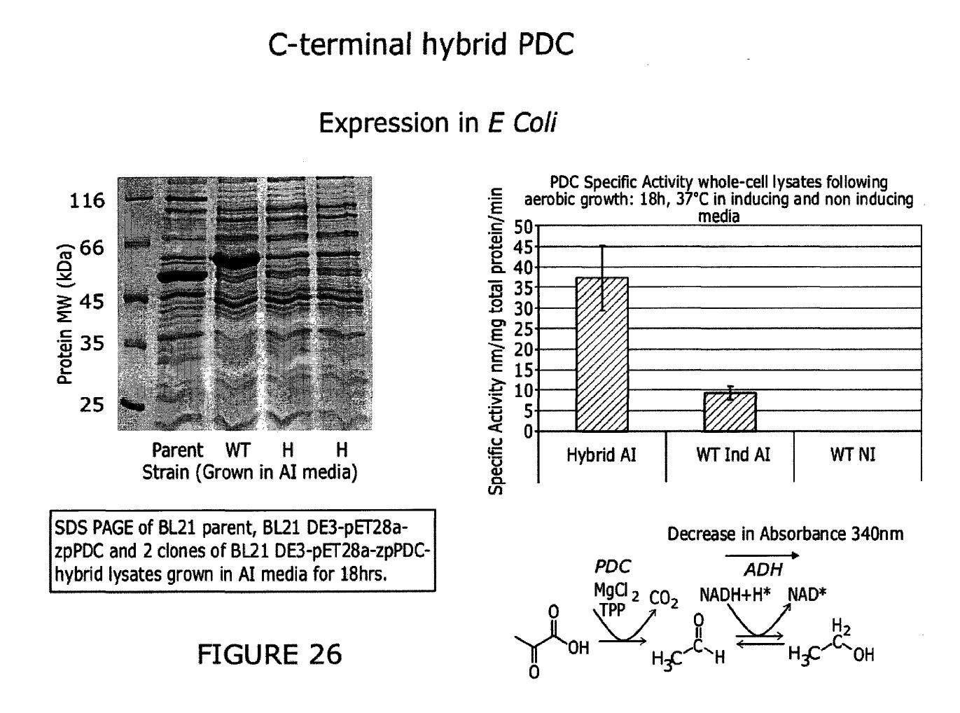

FIG. 26 shows expression of a C-terminal hybrid PDC in E. coli. The specific PDC activity of the hybrid was greater than that of the wild type PDC, following expressing at 37° C. We believe that this is due to tighter binding of TPP.

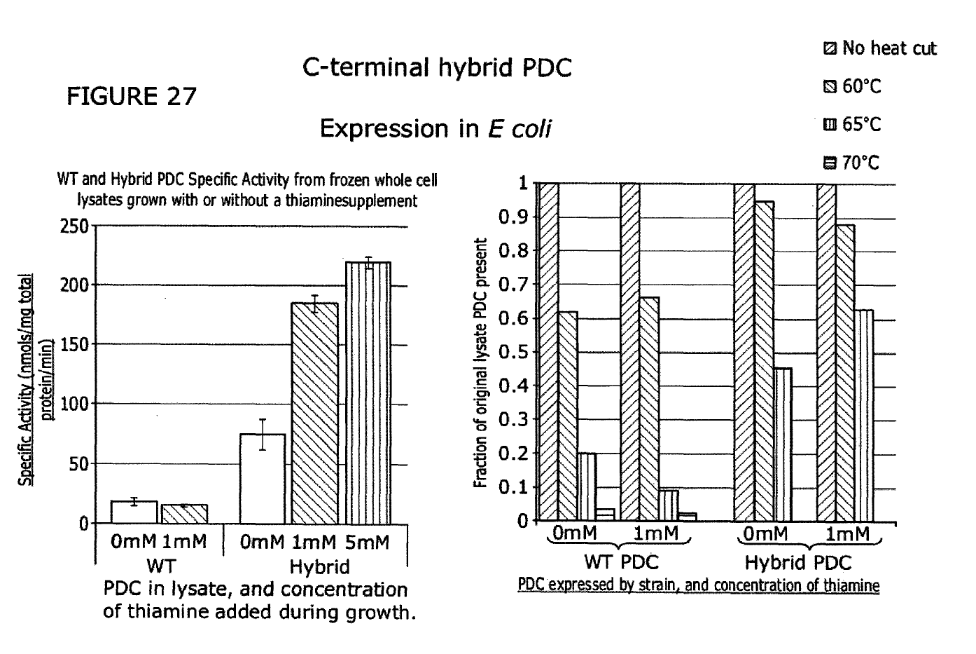

FIG. 27 (left hand panel) shows the effect of thiamine concentration on the specific activity of wild type PDC and a hybrid PDC. Again, the specific activity of the hybrid PDC is significantly more than that of the wild type PDC, even in the absence of thiamine. Also, the addition of thiamine (which will increase TPP) increases the specific activity of the hybrid PDC.

FIG. 27 (right hand panel) shows the specific PDC activity of wild type and hybrid PDC, following heating of the cell extract at various temperatures, as a percentage of the original PDC activity (i.e. that at no heat treatment). Intriguingly a higher % of the hybrid activity is retained, which suggests that the hybrid PDC is more thermostable per se.

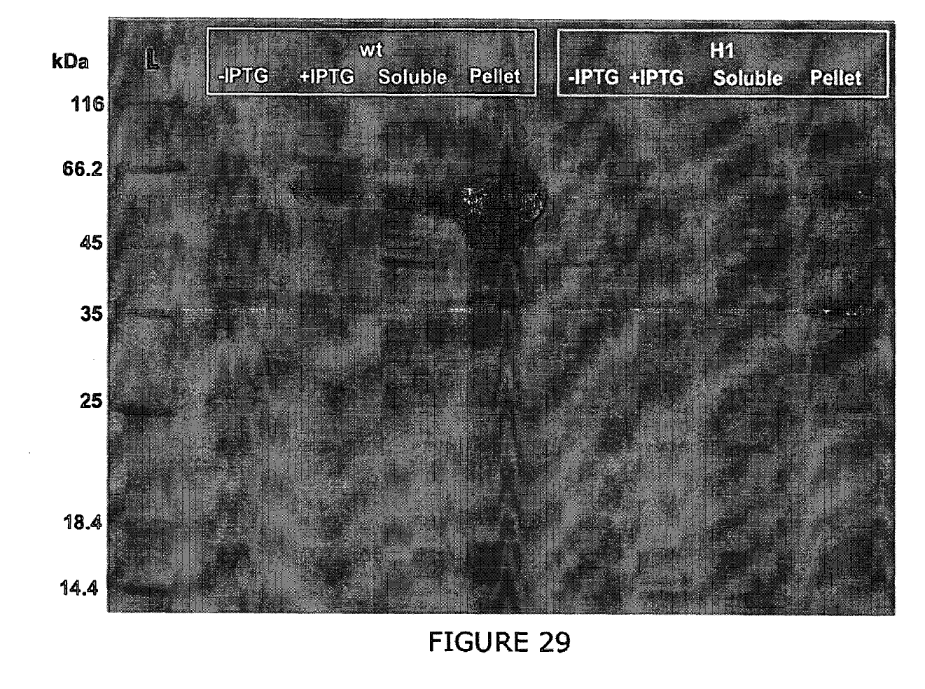

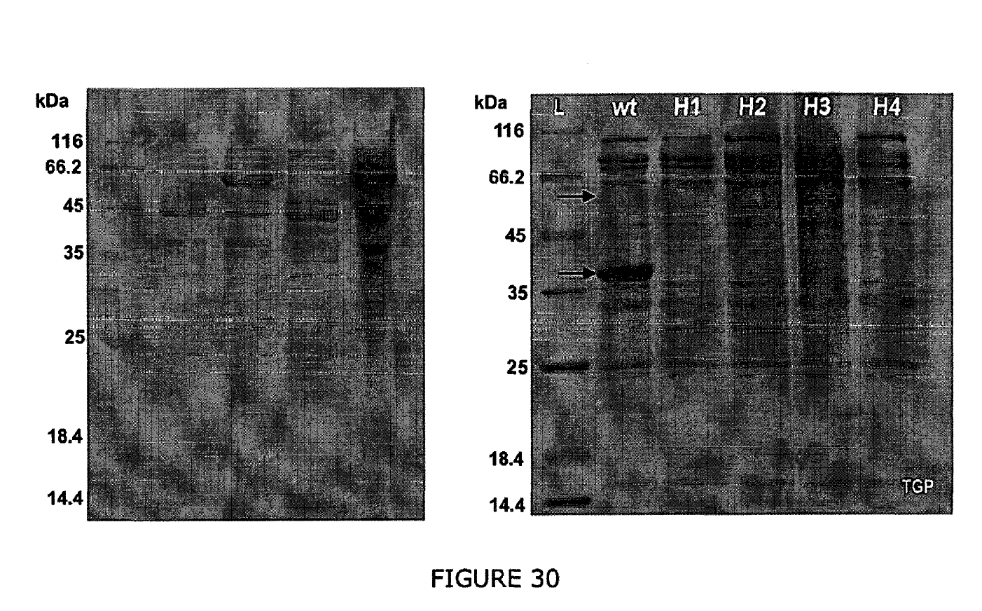

FIGS. 29 and 30 show expression of wild type and various hybrid PDCs.

### Example 3

### Thermostability of the Modified PDC Enzymes

### Experiments and Results

We tested the modified PDC enzymes from Example 2 for their stability upon folding at increased temperature. Of these, the most dramatic differences have been observed with the longer N terminal sequence N₂ and N₂C, which demonstrated a 7-8° C. improvement. N₁ and N₁C appeared to have poor stability (a problem with hybrid proteins). C alone was stable, and showed a less-marked improvement in thermostability.

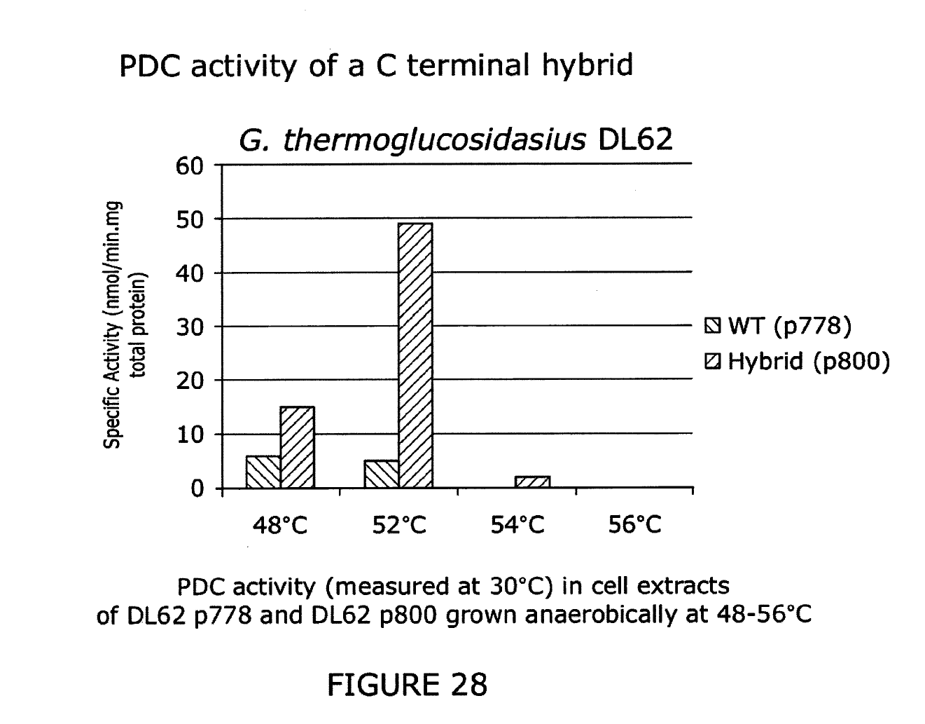

FIG. 28 shows the PDC activity of a C-terminal hybrid expressed in G thermoglucosidasius DL62 at various temperatures. The hybrid exhibits higher specific activity than the wild type PDC when expressed at high temperatures.

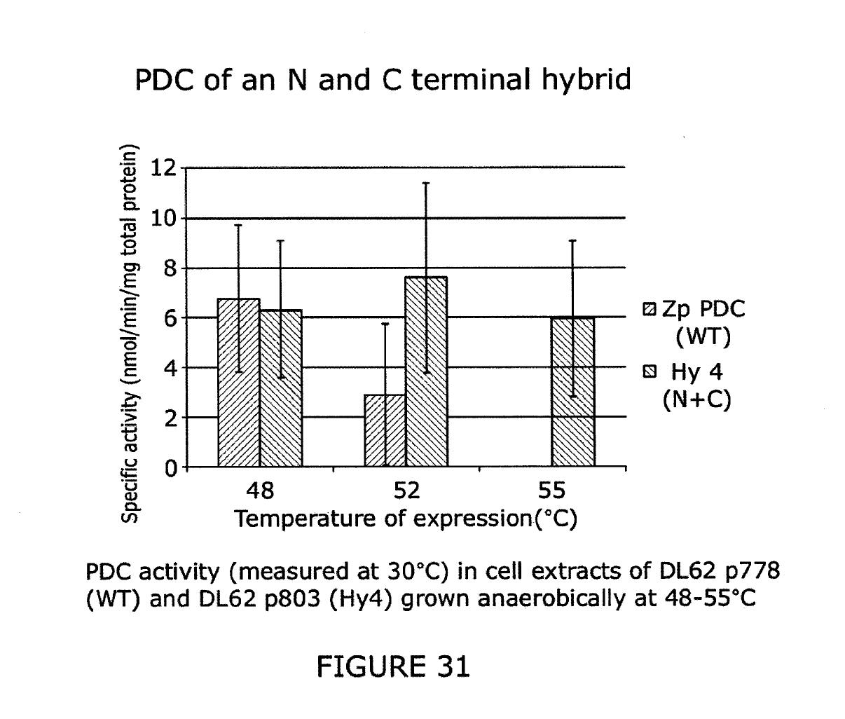

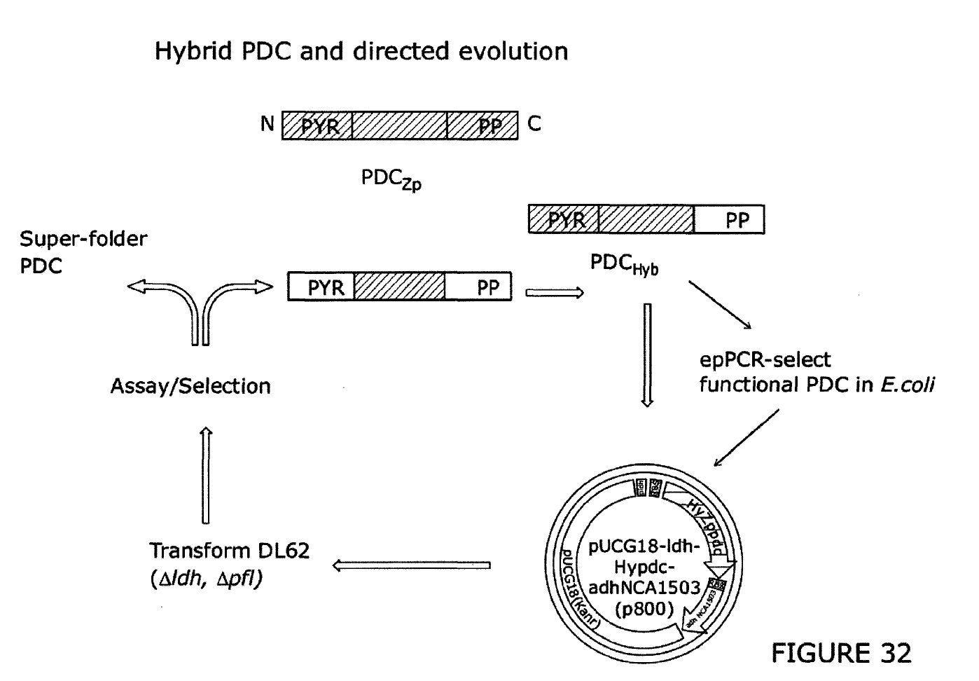

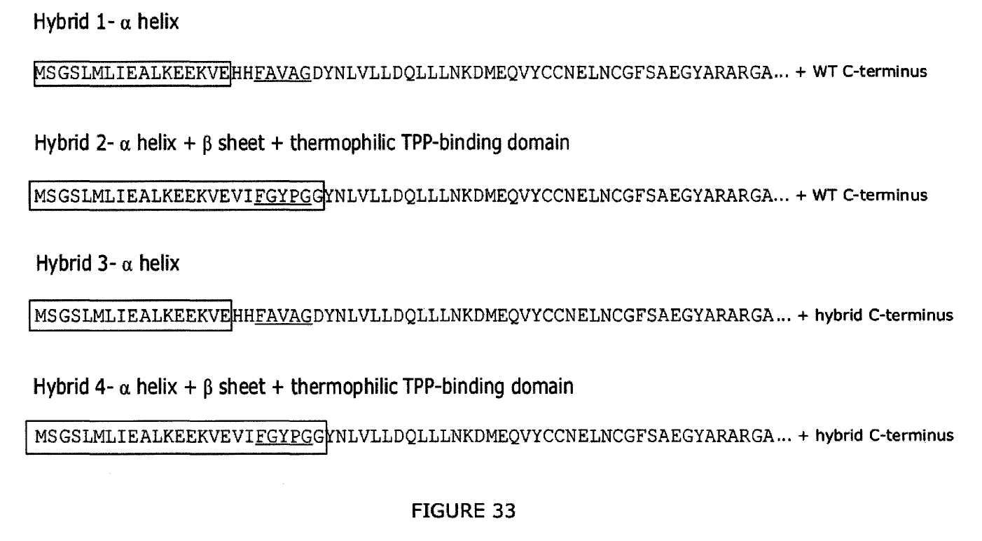

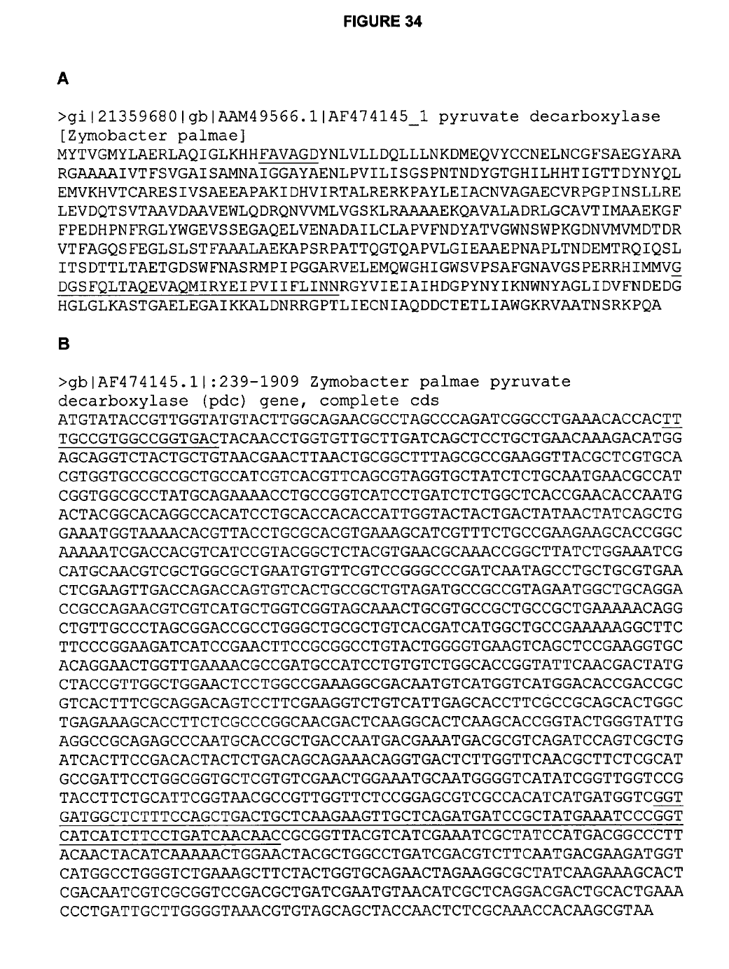

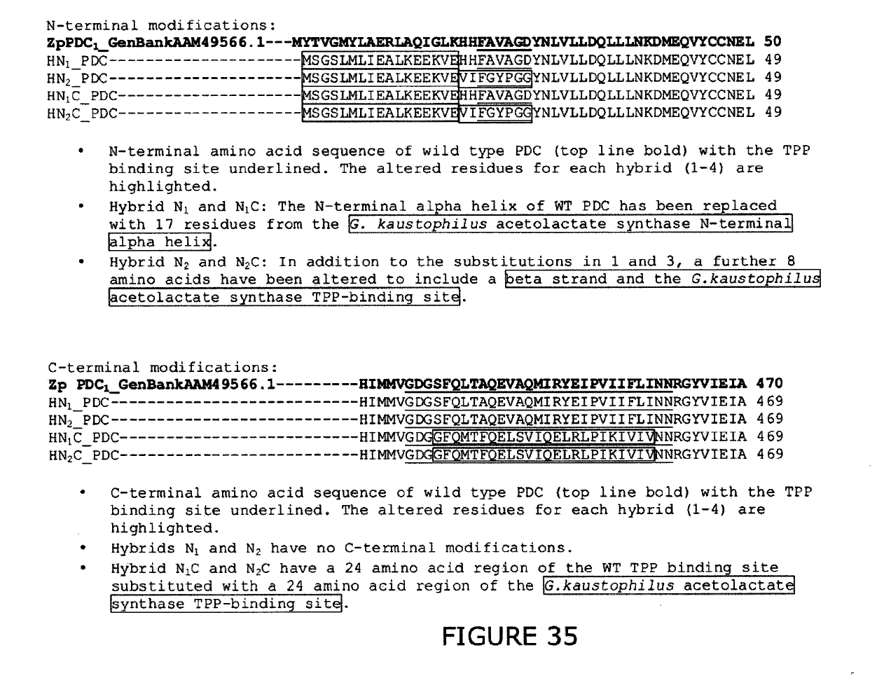

FIG. 31 shows the PDC activity of an N and C terminal hybrid PDC expressed in G. thermoglucosidasius DL62 at various temperatures. Again, the hybrid exhibits higher specific activity than the wild type PDC when expressed at high temperatures.

### Discussion

We have shown that it is possible to make a more thermostable PDC enzyme by replacing the TPP binding motif with an equivalent domain from a thermophilic protein. This is a major advance towards the production of a thermostable PDC enzyme that can be expressed in thermophilic bacteria, for example in continuous culture at higher temperatures.

Since the TPP binding motif at both the N- and C-termini is common in TPP containing proteins, these findings have generic implications for the broader family of keto-acid decarboxylases.

### Example 4

### Assaying PDC Activity

Genes capable of expressing either a unmodified or TPP binding site modified PDC are cloned into a vector capable of replicating in the desired thermophilic host eg they can be cloned into the vector pUCG18 behind a promoter capable of driving expression such as the Idh promoter, and transformed into Geobacillus thermoglucosidasius. Cells of the thermophile are then grown at different temperatures (eg 48° C., 50° C., 52° C., 54° C., 56° C. etc) under conditions which cause the gene to be expressed. Cells, harvested in logarithmic growth phase are then broken open (eg with a French Press) and the soluble cell extract is recovered for assaying PDC. PDC activity can be measured using the standard assay techniques at 30° C., a typical assay involving coupling the production of acetaldehyde (from pyruvate decarboxylation) to its reduction by an added alcohol dehydrogenase. The activity of the latter is measured in a spectrophotometer by following the oxidation of NADH at 340 nm.

A typical assay is as follows:

To calculate the background rate of NADH oxidation Abs₃₄₀ₙₘ is measured from a pre-warmed (30° C.) mix containing: 152 μl KOH-MES buffer, 6.6 μl 5 mM NADH, 1.4 μl 1500 U/mL ADH (Sigma-Aldrich) and 30 μl of protein sample in a 96 well plate (BD Falcon). Background Abs₃₄₀ₙₘ is measured during 5 minutes, at intervals of 20 seconds, in a Synergy HT microplate reader. To calculate actual PDC activity, 7 μl of 500 mM sodium pyruvate is added to the mix and Abs₃₄₀ₙₘ measured again over 5 minutes. PDC activity is quantified by measuring the change in Abs₃₄₀ₙₘ before and after addition of sodium pyruvate following the NADH coupled assay principle.

Results are the mean of triplicate assays. Enzyme specific activity values are obtained by dividing the enzyme activity by the total protein concentration. Pyruvate decarboxylase from baker's yeast S. cerevisiae (Sigma-Aldrich) is used as positive control at a final concentration of 0.6 units.

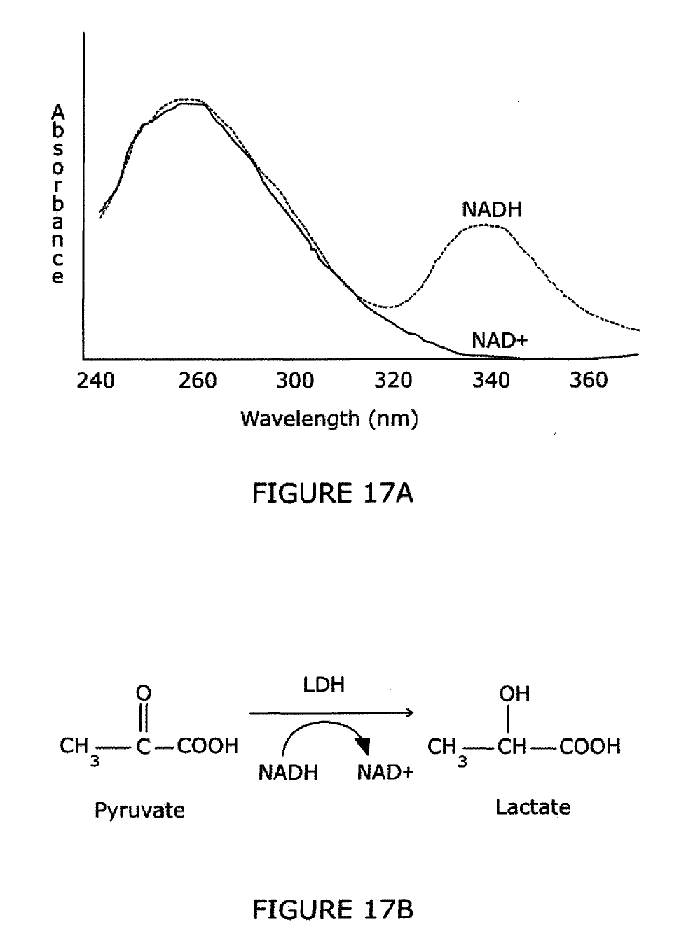

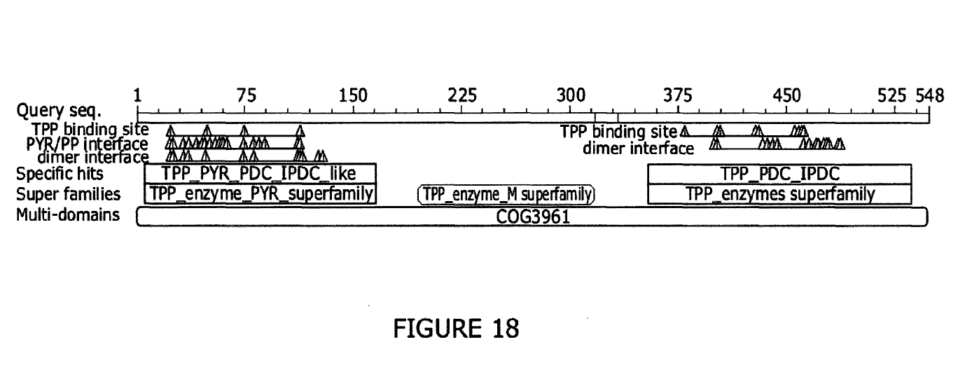

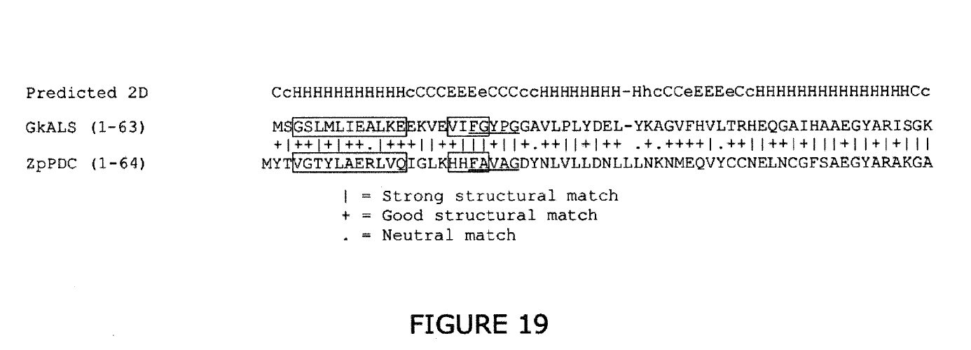

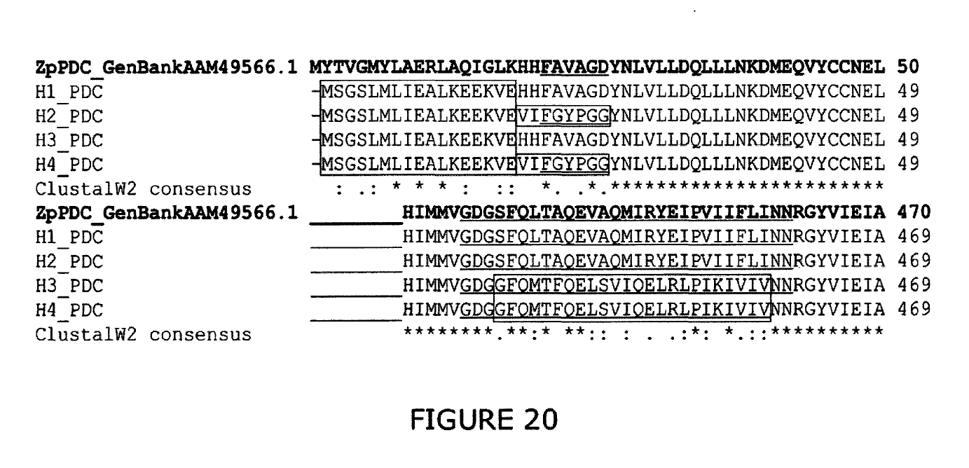

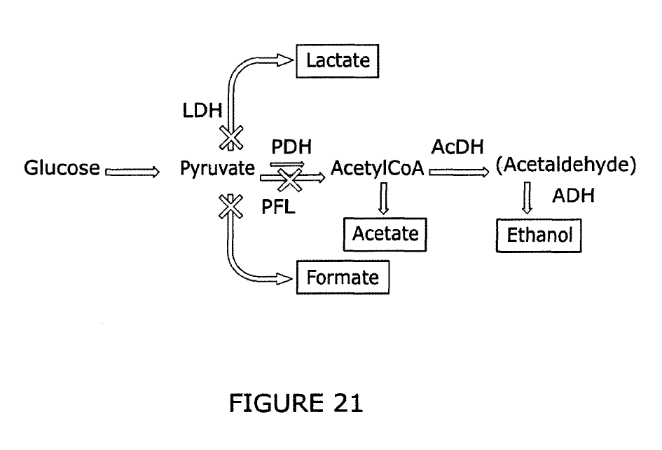

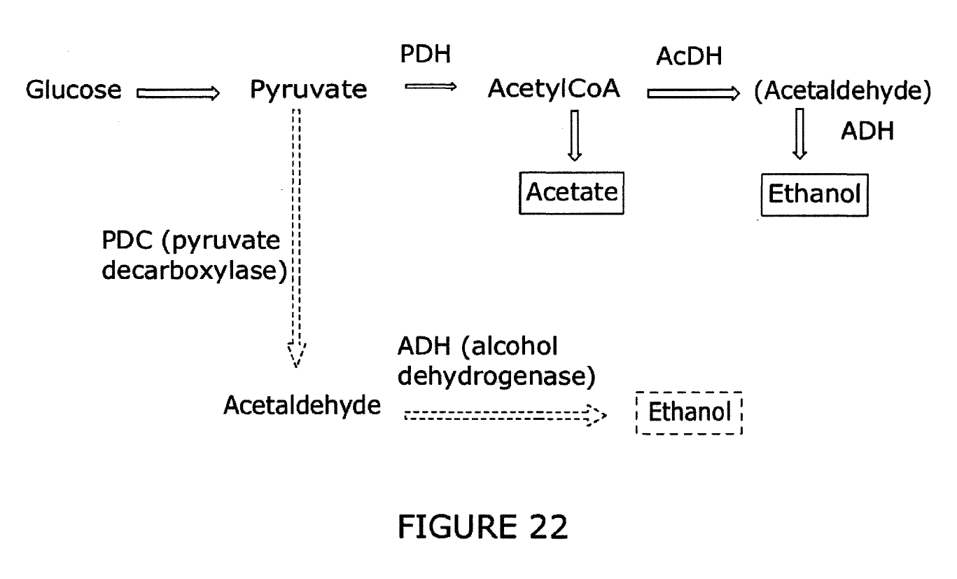

Typical results are shown in FIG. 17.

### SUMMARY OF EXAMPLES

Pyruvate decarboxylase from Zymomonas needs TPP and Mg²⁺ cofactors to fold in an active conformation.
TPP binding sites in Geobacillus kaustophilus acetolactate synthase have a “thermophilic” motif.
A hybrid enzyme comprised of Z. palmae PDC with a TPP binding site from G. kaustophilus ALS is active and shows evidence of folding at higher temperatures than the parent enzyme.
Growth and PDC activity of G thermoglucosidasius DL62 expressing the hybrid enzyme indicates that it is possible to create a viable strain in which PDC and ADH form the sole fermentation pathway.

### REFERENCES

Arnold, F. H. & A. A. Volkov. (1999) Directed evolution of biocatalysts. Current Opinion in Chemical Biology. 3, 54-59.
Atsumi S, Hanai T & Liao J C (2008) Nature 451:86-89.
Brandt A, Hallett J P, Leak D J, Murphy R J & Welton T (2010) The effect of the ionic liquid anion in the pretreatment of pine wood chips. Green Chem., 12, 672-679.
Brandt A, Ray M, To T Q, Leak D J, Murphy R J & Welton T (2011) Ionic liquid pretreatment of lignocellulosic biomass with ionic liquid water mixtures Green Chem 13 2489-2499.
Conway, T., Osman, Y. A., Konnan, J. I., Hoffmann, E M. & Ingram, L. O. Promoter and nucleotide sequences of the Zymomonas mobilis pyruvate decarboxylase J. Bacteriol. 169 (3): 949-954 (1987) (sequence corrected 2002).
Cripps R E, Eley K, Leak D J, et al (2009) Metab Eng 11:398-408.
Crowhurst N A and Leak D J, (2010) Biobutanol: A better biofuel?, Int Sugar J, 112, 64-73.
de la Plaza, M., Fernandez de Palencia, P., Pelaez, C. & Requena, T. Biochemical and molecular characterization of alpha-ketoisovalerate decarboxylase, an enzyme involved in the formation of aldehydes from amino acids by Lactococcus lactis. FEMS Microbiol. Lett. 238 (2), 367-374 (2004) gi|51870502|emb|CAG34226.1|).
Farrell A E, Plevin R J, Turner B T, Jones A D et al (2006) Science 311: 506-508.
Hoppner, T C & Doelle H W (1983) “Purification and kinetic characteristics of pyruvate decarboxylase and ethanol dehydrogenase from Zymomonas mobilies in relation to ethanol production,” European Journal of Applied Microbiology and Biotechnology, 17, 152-157.
Ingram L O, Conway T, Clark D P et al 1987 App Env Microbiol 53: 2420-2425.
Mezzasalma et al, J Biomol Screening, 2007, April; 12(3):418-428.
Pohl, M., J. Grotzinger, A. Wollmer and M. R. Kula. (1994) Reversible Dissociation and Unfolding of Pyruvate Decarboxylase from Zymomonas-Mobilis. European Journal of Biochemistry. 224, 651-661
Ragauskas, A J, Williams, C K, Davison, B H, et al (2006) Science 311: 484-489.
Ray M J, Leak D J, Spanu P D and Murphy R J (2010) Brown rot fungal early stage decay mechanism as a biological pretreatment for softwood biomass in biofuel production Biomass and Bioenergy 34: 1257-1262.
Shaw A J, Podkaminer K K, Desal S G et al (2008) PNAS 105: 13769-13774.
Taylor, M P, Esteban, C D, Leak, D J, (2008) Development of a versatile shuttle vector for gene expression in Geobacillus spp., Plasmid 60: 45-52.
Taylor M P, van Zyl L, Tuffin I M, Leak D J, Cowan D A (2011) Genetic tool development underpins recent advances in thermophilic whole-cell biocatalysts., Microb Biotechnol, 4: 438-448.
Thompson A H, Studholme D J, Green E M and Leak D J (2008) Expression of pyruvate decarboxylase in Geobacillus spp Biotech Lett 30:1359-1365.
van Haveren J, Scott E L & Sanders J (2008) Biofuels. Bioprod. Bioref. 2: 41-57.
Yang B & Wyman C E (2008) Biofuels, Bioprod. Bioref. 2 26-40.
Zhou S, Davis F C & Ingram L O (2001) Appl. Env. Microb. 67:6-14.

1. A method of improving the folding of an enzyme comprising a thiamine pyrophosphate (TPP) binding domain, the method comprising:
providing a nucleic acid encoding the enzyme comprising a TPP binding domain, in which one or more of the TPP binding domains in the enzyme monomer are replaced with a TPP binding domain from a thermostable TPP-binding protein, and
expressing the nucleic acid under conditions that allow expression and folding of the enzyme.

2-3. (canceled)

4. A method according to claim 1, further comprising determining whether the enzyme has folded.

5. A method according to claim 4, wherein determining whether the enzyme has folded comprises assessing a biological activity of the enzyme.

6. A method according to claim 5, wherein the enzyme is a keto-acid decarboxylase and the enzymic activity is keto-acid decarboxylase activity.

7. A method according to claim 1, wherein the enzyme that comprises a TPP binding domain is any of a keto-acid decarboxylase enzyme, a pyruvate decarboxylase, a keto-isovalerate decarboxylase, an alpha-ketoacid dehydrogenase, a branched chain amino acid dehydrogenase, a transketolase, a 2-hydroxyphytanoyl-CoA lyase, an alpha-ketoacid ferredoxin oxidoreductase, a glyoxylate carboligase, an oxalyl-CoA decarboxylase, an acetolactate synthase, an alpha-ketoacid oxidase, a sulfoacetaldehyde acetyltransferase, a 2-succinyl-5-enolpyruvyl-6-hydroxy-3-cyclohexene-1-carboxylate synthase, a pyruvate synthase, an epi-inositol hydrolase, a malonic semialdehyde oxidative decarboxylase, a pyruvate:flavodoxin oxidoreductase, a 3D-(3,5/4)-trihydroxycyclohexane-1,2-dione hydrolase, a 2-oxoglutarate synthase, a 2-succinyl-6-hydroxy-2,4-cyclohexadiene-1-carboxylic acid synthase, a phosphonopyruvate decarboxylase, a sulfopyruvate decarboxylase, a phenylglyoxylate:acceptor oxidoreductase, and a myo-inositol catabolism protein lolD.

8. A method according to claim 1, wherein a cell is transformed with the nucleic acid, and the enzyme is expressed in the cell.

9. A method according to claim 8, wherein the cell is a thermophilic cell.

10. A method according to claim 9, wherein the thermophilic cell is a Geobacillus sp., such as a Geobacillus thermoglucosidasius cell or a Geobacillus kaustophilus cell.

11. A method according to claim 1, wherein the enzyme comprising a TPP binding domain is derived from a mesophile.

12. A method according to claim 1, wherein the enzyme comprising a TPP binding domain is derived from bacteria of the genus Zymomonas, such as Zymomonas mobilis or Zymomonas palmae.

13. A method according to claim 1, wherein the one or more TPP binding domains of the enzyme comprising a TPP binding domain are located at the N-terminus and/or C-terminus of the enzyme.

14. A method according to claim 13 wherein the TPP binding domain at the N-terminus is an amino acid sequence that corresponds to the N-terminal TPP binding domain of Zymomonas palmae pyruvate decarboxylase.

15. A method according to claim 13 wherein the TPP binding domain at the C-terminus is an amino acid sequence that corresponds to the C-terminal TPP binding domain of Zymomonas palmae pyruvate decarboxylase.

16. A method according to claim 1, wherein the thermostable TPP-binding protein is derived from a thermophilic organism.

17. A method according to claim 1, wherein the thermostable TPP-binding protein is an acetolactate synthase.

18. A method according to claim 1, wherein the TPP binding domain of the thermostable TPP-binding protein is at the N-terminus or C-terminus of the thermostable TPP-binding protein.

19. A method according to claim 18 wherein the TPP binding domain at the N-terminus of the thermostable TPP-binding protein is an amino acid sequence that corresponds to the N-terminal TPP binding domain of acetolactate synthase from G. kaustophilus.

20. A method according to claim 18 wherein the TPP binding domain at the C-terminus of the thermostable TPP-binding protein is an amino acid sequence that corresponds to the C-terminal TPP binding domain of acetolactate synthase from G. kaustophilus.

21. A method according to claim 1, comprising replacing one or more TPP binding domains in a Zymomonas pyruvate decarboxylase with an N-terminal and/or C-terminal TPP binding domain of acetolactate synthase from G. kaustophilus.

22. A method according to claim 1, further comprising isolating the expressed and folded enzyme.

23. A method of improving the thermostability of an enzyme comprising a TPP binding domain, the method comprising replacing one or more TPP binding domains in the enzyme with a TPP binding domain from a thermostable TPP-binding protein.

24-28. (canceled)

29. A method according to claim 23, wherein the one or more TPP binding domains of the enzyme comprising a TPP binding domain are located at the N-terminus and/or C-terminus of the enzyme.

30. A method according to claim 23, wherein the TPP binding domain from a thermostable TPP-binding protein is derived from a thermophilic organism.

31. A method according to claim 23, wherein the nucleic acid encodes a keto-acid decarboxylase in which one or more TPP binding domains in the keto-acid decarboxylase enzyme are replaced with an N-terminal and/or C-terminal TPP binding domain of acetolactate synthase from Geobacillus kaustophilus.

32. (canceled)

33. A method according to claim 23, wherein the enzyme is a keto-acid decarboxylase and the enzymic activity is keto-acid decarboxylase activity.

34. A method according to claim 23 wherein the enzyme is a pyruvate decarboxylase enzyme.

35-77. (canceled)
